(19) United States
(12) Patent Application Publication
(10) Pub. No.: US 2008/0077146 A1
(43) Pub. Date: Mar. 27, 2008

(54) ANNULUS CUTTING TOOLS AND METHODS

(75) Inventors: Weston Pernsteiner, Marquette, MI (US); Thomas Kilpela, Marquette, MI (US); Qi-Bin Bao, Marquette, MI (US)

Correspondence Address: FITCH EVEN TABIN AND FLANNERY, 120 SOUTH LA SALLE STREET, SUITE 1600, CHICAGO, IL, 60603-3406, US

(73) Assignee: PIONEER SURGICAL TECHNOLOGY, INC., Marquette, MI (US)

(21) Appl. No.: 11/859,476

(22) Filed: Sep. 21, 2007

Related U.S. Application Data
(60) Provisional application No. 60/846,520, filed on Sep. 21, 2006.

(51) Int. Cl. A61B 17/3213

(52) U.S. Cl. 606/79; 606/170; 606/172; 606/181

(57) ABSTRACT

The present invention relates to tools and methods for cutting the annulus of a spinal disc. The cutting tools of the invention may be cannulated for use in conjunction with a guide wire. A stop surface of the cutting tool is configured to contact the annulus during cutting to prevent further longitudinal movement of the surgical blade(s) into the annulus, thus limiting the size of the incision(s).

### CROSS-REFERENCE TO RELATED APPLICATION

This application claims the benefit of U.S. Provisional Application 60/846,520, filed on Sep. 21, 2006, the specification of which is incorporated herein in its entirety.

### FIELD OF THE INVENTION

The present invention relates to tools and methods for cutting the annulus of a spinal disc.

### BACKGROUND OF THE INVENTION

The most common orthopedic condition for which professional medical treatment is sought is lower back pain. Although many factors may be responsible for causing lower back pain, a principal factor is damage or degeneration of an intervertebral spinal disc resulting in impingement on the nerve system, specifically the spinal cord, located within the spine. Such impingement may result in, for instance, loss of mobility, urinary and fecal incontinence, and sciatica or pain experienced in the extremities.

Damage to or degeneration of a spinal disc can result from a number of factors such as abuse or age. The disc itself is composed primarily of an annulus and a nucleus contained therein. The annulus is a fibrous annular piece that connects to the adjacent vertebrae and contains the nucleus, which is in turn a gel-like viscous material capable of shock absorption and flowable to permit poly-axial rotation and resilient compression of the vertebrae and spine. Most frequently, disc degeneration results from damage occurring to the annulus such that the flowable nucleus material may leak or seep out of the annulus. Disc degeneration also can occur in other ways, such as by being deprived of nutrient flow leading to a dried disc susceptible to damage. Because the nuclear material is flowable, extensive damage to the annulus is not necessary for leakage to occur.

Currently, approaches to treatment of spinal problems directly affecting the spinal cord are numerous. For instance, immobilization and high doses of corticosteroids may be employed. The dominant surgical procedures for treatment of these problems are spinal fusion and discectomy. Fusion is a method where adjacent vertebrae are immobilized so that they permanently fuse to each other by having bone growth between and to the vertebrae, while discectomy involves removal of a portion or an entirety of a spinal disc.

However, the current practice of each of these procedures typically has certain limitations. With fusion, making a portion of the spine generally rigid produces a reduction in mobility, and drastically alters normal load distribution along the spinal column. Due to these factors, the non-fused portions of the spine experience stress and strain that are significantly increased over normal physiological motions. The increased stress and strain on the non-fused portions may lead to accelerated disc degeneration of the non-fused portions, particularly the adjacent levels of the spine.

Discectomy is effective for relieving sciatic pain by removing the damaged or herniated disc tissue compressing the spinal nerves. However, current discectomy often may lead to a reduction of the disc space between adjacent vertebrae, as well as instability in the affected portion of the spine. Such long-term effects with current discectomy often result in further surgery several years after the initial discectomy surgery.

In an alternative spinal surgery, a disc arthroplasty restores or reconstructs the disc using a prosthesis to replace a portion or entirety of the damaged disc. The primary objective of disc arthroplasty is to restore or maintain the normal disc anatomy and functions, while addressing and treating the causes of the pain. However, prosthetic disc implants have problems due to the complexity of the natural disc structure and biomechanical properties of a natural spinal disc. As used herein, the term natural refers to normal tissue including portions of the spine and the disc.

Two types of prostheses for disc arthroplasty are currently believed to merit further development by medical science and research. One type is a total disc prosthesis, or TDP, where the entire spinal disc is replaced after radical discectomy. A typical TDP includes structures that attempt to together mimic the properties of a natural disc.

The other type is a disc nucleus prosthesis, or DNP, that is used to replace only the nucleus of a spinal disc after a nucleotomy while retaining the annulus of the disc and, possibly, the end plates intact. As discussed above, failure of the natural disc does not require extensive damage to the annulus. An undamaged annulus, however, would often be capable of retaining a non-flowing prosthetic nucleus. Implantation of a DNP involves making a small incision in the annulus, clearing of the natural nucleus from the annulus through the procedure known as nucleotomy, and inserting the DNP through, and then within, the annulus. Accordingly, DNPs are typically smaller and require less extensive surgery than TDPs while still mimicking some of the biomechanical properties of a natural intervertebral disc.

Implantation of DNPs require an incision in the annulus that forms an opening of sufficient size for the DNP to be inserted therethrough. It is also important that the incision in the annulus is kept as small as possible to minimize the potential for the implant to back out through the incision. The annulus itself is used to at least aid in maintaining the implant within the nuclear space. This permits the DNP to sit in the intervertebral space without anchors that violate the end plates of the vertebrae. As the annulus does not heal well and suturing the annulus is difficult due to its tissue properties, once the incision is too large, the ability of the annulus to retain the implant is diminished if not eliminated. Accordingly, precision in cutting an incision to a size that forms the opening so that it is just big enough to fit the DNP therethrough is important to keep the DNP from backing out of the nuclear space.

Generally, surgeons use conventional scalpels to create incisions in the annulus for discectomy or disc arthroplasty surgeries. The risk of creating an incision in the annulus that is too large is increased with conventional scalpels. Conventional scalpels have a handle and a single attachment portion for a fixed or disposable surgical blade. Conventional scalpels are commercially available in a variety of standardized handles as set by the International Organization for Standardization (ISO). Generally, conventional surgical blades used with scalpels have an inclining, slightly curved cutting edge. However, conventional scalpels do not provide means of controlling the cutting length and/or shape of the incision. Thus, a need exists for a cutting tool that is specifically adapted for cutting an annulus, and an annulus cutting tool that is operable to create a precisely sized incision in the annulus and particularly to create an incision of desired length in the wall of the annulus.

### SUMMARY OF THE INVENTION

In accordance with the embodiments illustrated herein, annulus cutting tools and methods for cutting the annulus of a spinal disc in a controlled manner to provide precisely sized incisions. Commercially available or custom surgical blades may be used with the cutting tools of the invention. Generally, the blade mounting portions for the tools described hereinafter are adapted to be used with commercial surgical blades so that the tool mounting portions thereof do not need to be customized for being mounted to the tools herein. If the blades are to be customized, it is preferred that these custom blades still include the standard tool mounting portions of commercial blades.

In one aspect of the invention, an annulus cutting tool is provided which is configured for the attachment of two surgical blades in the same orientation. More particularly, the tool includes an elongate shaft and a mounting portion toward the distal end thereof. The mounting portion is configured so that a plurality of surgical blades may be mounted to the cutting tool shaft such that the surgical blades are separated by a fixed distance. The mounting portion preferably includes a stop surface at the distal end of the tool at a predetermined position relative to and extending between the surgical blades. During surgery, the stop surface of the cutting tool can contact the annulus to substantially keep inclined cutting edges of the surgical blades from continuing to cut and enter the annulus beyond a predetermined distance. Accordingly, if the stop surface is located closer to the end of the cutting edge, then the inclined cutting edge will only cut into the annulus wall for the small length thereof that extends beyond the stop surface, thus forming an incision of only a small length. By contrast, if the stop surface is located further back along the length of the inclined cutting edge, then a greater length of the inclined cutting edge will be exposed beyond the stop surface and be able to be used for cutting into the annulus wall, thus forming an incision of a longer length in the annulus wall. Therefore, the surgeon may use the present cutting tool to create parallel incisions of a desired predetermined length in the wall of the annulus.

After a first set of parallel incisions has been made, the blades are withdrawn from the annulus wall for a making a second set of incisions therewith to form a square-shaped opening, for example, with equal length sides. Alternatively, the fixed distance between the blades can be longer or shorter than the length of the incisions the blades are preset to cut via the location of the stop surface relative thereto so that another tool is required having blades set for appropriately sized incisions to interconnect the first set of incisions for forming the opening with a rectangular shape. If the tool is provided with an adjustable stop surface as described further hereinafter, then the same tool can be used for both cutting procedures with an appropriate adjustment made after the first set of incisions has been made with the blades withdrawn from the annulus wall and before the second cutting procedure.

In a preferred form, the shaft of the cutting tool may also be cannulated such that the cutting tool may be used in conjunction with a guide wire.

In another aspect, the blade mounting portion is configured such that two substantially identical surgical blades, and specifically having identical mounting portions thereof, may be mounted on the annulus cutting tool in substantially opposite orientations. In this manner, the blades have their cutting edges facing in substantially opposite directions. This configuration of the blades allows the surgeon to advance the blades into the annulus wall to make the initial cuts with the blades, and then turn the shaft to cut an opening in the annulus wall with the surgical blades without having to withdraw the blades from the annulus and insert the blades for a second cutting operation therewith.

In another form, the blade mounting portion is configured such that the surgical blades can be mounted in either of the aforementioned orientations. This allows the blades to be mounted so that the cutting edges of the blades face the same direction or opposite directions to form openings in the annulus wall either of the above-described methods.

In another form, the blade mounting portion is configured to hold a single surgical blade. This embodiment is particularly useful when the cutting tool is cannulated and used in conjunction with a guide wire to create a single incision at a predetermined distance from the guidewire. Preferably, the surgical blade can also be curved for generally arcuate incisions.

In another aspect, the mounting portion is configured to hold four surgical blades. The four surgical blades can be advanced into the annulus wall for forming the four sides of an opening simultaneously therein without the need for a second cutting operation or turning of the tool handle.

In another aspect, the mounting portion includes an adjustable stop portion adjacent thereto. The adjustable stop portion allows the length of the incision to be made with a particular size of blade to be selectively varied. In one form, the surgeon may adjust the stop portion via operation of an actuator such as by rotating an adjustment knob so that a desired predetermined length cut in the annulus is made with the blade or blades.

In another form, the mounting portion includes a detent mechanism that releasably holds the adjustable stop portion at a select position chosen from multiple predetermined positions.

In another aspect of the invention, a cannulated cutting tool configured to hold a single surgical blade is provided for use with a guide wire.

### BRIEF DESCRIPTION OF THE DRAWINGS

In the drawings, FIGS. 1A and 1B show elevational views of surgical blades. The surgical blade of FIG. 1A is of conventional construction and has a linear inclined cutting edge. The surgical blade of FIG. 1B has a curved cutting edge.

FIG. 2 is a perspective view of an annulus cutting tool having a distal blade mounting portion configured to hold two surgical blades in a same orientation in accordance with the present invention. FIG. 2A is a perspective view of an alternative embodiment of the shaft of the annulus cutting tool of FIG. 2;

FIG. 3 is an enlarged top perspective view of the annulus cutting tool of FIG. 2 showing blade holding grooves and locating indentations of the blade mounting portion.

FIG. 4 is an enlarged left-side, elevational view of the annulus cutting tool of FIG. 2.

FIG. 5 is an enlarged right-side, elevational view of the annulus cutting tool of FIG. 2.

FIG. 6 is an enlarged, perspective view of an annulus cutting tool with two surgical blades mounted thereon in the same orientation.

FIG. 7 is a top perspective view of another cutting tool having a modified blade mounting portion with reversely configured blade locating indentations.

FIG. 8 is an enlarged left-side, perspective view of the blade mounting portion of FIG. 7 showing one of the reversely configured indentations.

FIG. 9 is an enlarged right-side, elevational view of the blade mounting portion of FIG. 7 showing the other indentation reversely configured from the indentation of FIG. 8.

FIG. 10 is an enlarged left-side, perspective view of the blade mounting portion of FIG. 7 with two surgical blades mounted thereon in opposite orientations.

FIG. 11 is a perspective view of another cutting tool having a blade mounting portion that allows the blades to be held in different orientations.

FIG. 12 is a left-side perspective view of the blade mounting portion of FIG. 11 showing one of the indentations having a V-shaped locating surface.

FIG. 13 is a right-side perspective view of the blade mounting portion of FIG. 12 showing the V-shaped surface of the other indentation.

FIG. 14 is a perspective view of the blade mounting portion of FIG. 7 with two surgical blades mounted thereon in opposing orientations.

FIG. 15 is a perspective view of another annulus cutting tool having a blade mounting portion configured for holding a single surgical blade.

FIG. 16 is an enlarged side perspective view of the blade mounting portion of the cutting tool of FIG. 15 showing a single blade locating portion and a single blade holding portion.

FIG. 17 is a perspective view of another annulus cutting tool having a shaft assembly configured to form a mounting portion that holds four surgical blades.

FIG. 18 is an enlarged perspective view of an internal shaft of the shaft assembly of FIG. 17 showing an end portion thereof configured to hold two surgical blades.

FIG. 19 is an enlarged perspective view of an outer shaft of the shaft assembly of FIG. 17 showing a split end portion thereof configured to hold two surgical blades.

FIG. 20 is an enlarged perspective view of the annulus cutting tool of FIG. 17 showing sliding of the internal and outer shafts relative to each other.

FIG. 21 is a perspective view of another annulus cutting tool having an adjustable stop portion.

FIG. 22 is a perspective view of an outer shaft of the annulus cutting tool of FIG. 21.

FIG. 23 is a perspective view of an inner shaft assembly of the annulus cutting tool of FIG. 21 having the stop portion at the distal end thereof.

FIG. 24 is an enlarged perspective view of the mounting portion and showing the adjustable stop portion adjacent thereto.

FIG. 25 is a perspective view of another embodiment of a cutting tool with an adjustable stop portion using a detent mechanism.

FIG. 26 is a perspective view of an outer shaft of the cutting tool of FIG. 25 showing detent balls of the detent mechanism.

FIG. 27 is a perspective view of the inner shaft of the cutting tool of FIG. 25 showing grooves of the detent mechanism.

FIG. 28 is an enlarged view of the detent mechanism showing a detent ball received in one of the grooves.

FIG. 29 is an enlarged fragmentary view of the blade mounting portion and the adjacent adjustable stop of the cutting tool of FIG. 25.

FIG. 30 is a perspective view of the cutting tool of FIG. 2 with two custom surgical blades mounted thereon.

FIG. 31 is an enlarged perspective view of the two custom surgical blades showing V-shaped cutting edges thereof.

FIG. 32 is a perspective view of another embodiment of a cannulated cutting tool having a mounting portion configured to hold one surgical blade.

FIG. 33 is an enlarged left-side fragmentary view of the mounting portion.

FIG. 34 is an enlarged right-side fragmentary view of the mounting portion showing an indented locating surface thereof.

FIG. 35 is an enlarged fragmentary top view of the mounting portion showing an end bar portion thereof offset from the tool shaft.

FIG. 36 is an enlarged right-side fragmentary view of the cutting tool of FIG. 30 with a surgical blade mounted thereon.

FIG. 37 is an enlarged left-side, perspective view of another embodiment of a cutting tool having an elongated stop portion.

### DETAILED DESCRIPTION OF THE PREFERRED EMBODIMENTS

In order to insert a spinal implant, such as an artificial disc, for replacing a nucleus of a natural spinal disc between adjacent vertebrae, a carefully sized incision must be made in the annulus of a spinal disc. It is important that the incision be sized appropriately so that the implant does not back out after insertion. The annulus cutting tools of the present invention provide a method of cutting an opening in the annulus to the dimensions desired.

Commercially available, detachable surgical blades may be used in the cutting tools of the invention. Because detachable surgical blades are commercially sold with a variety of shaped cutting edges, including inclining, declining, curved, and linear cutting edges, with a variety of cutting edge lengths, surgeons may vary the types of surgical blades used in the cutting tools of the invention based on personal preference or as required by the procedure to be undertaken and the anatomy of a particular patient.

Many different surgical blades known in the art may be used in the cutting tools of the invention. Because scalpel handles known in the art are standardized, surgical blades have standard features so that the blades can be interchangeably used with a variety of standardized handles. These generic features are illustrated in FIGS. 1A and 1B. It should be noted that FIGS. 1A and 1B are not intended to limit the types of surgical blades used with the invention to the blade embodiment depicted but are merely meant to illustrate the general features of surgical blades known in the art in order to demonstrate the ease with which a variety of surgical blades may be used with the cutting tools of the invention.

As generally known in the art and as shown in FIGS. 1A and 1B, a surgical blade 10 has a proximal end portion 12 and a distal end portion 14 with a longitudinal axis 16. A cutting edge 18, with a length L1 as measured from the distal tip 28 to the proximal tip 30 of the cutting edge, is located at the distal end portion 14 of the surgical blade 10. The length and shape of the cutting edge 18 may vary and is selected depending on the preference of the surgeon and of the needs of a particular surgery. FIG. 1A shows a surgical blade with a linear cutting edge that is inclined relative to the axis 16 while FIG. 1B shows a surgical blade with a curved cutting edge. Generally, surgical blades in the art have an inclining or declining cutting edge 18, although any type of cutting edge may be used. Surgical blades in the art may have a curved cutting edge, a straight edge, or a combination thereof. Although not depicted in FIGS. 1A and B, surgical blades in the art may have more than one cutting edge. The surgical blade 10 also has a tool mounting portion 19 including a rear abutment surface 20 and an elongate slot 22 configured to engage a mounting portion 109 of the cutting tool for attaching the surgical blade to the cutting tool, as will be described in more detail below. As shown, the slot 22 has varying width portions including a proximal end portion having a width W1 which is larger than the width W2 of the distal end portion 26 of the elongate slot 22.

Custom surgical blades may also be used with the cutting tools of the invention, depending on surgeon preference, as long as the custom blades have the same tool mounting portion 19 including the abutment surface 20 and elongate slot 22 which allow the custom blades to be mounted in a releasable fixed orientation on the cutting tools of the invention.

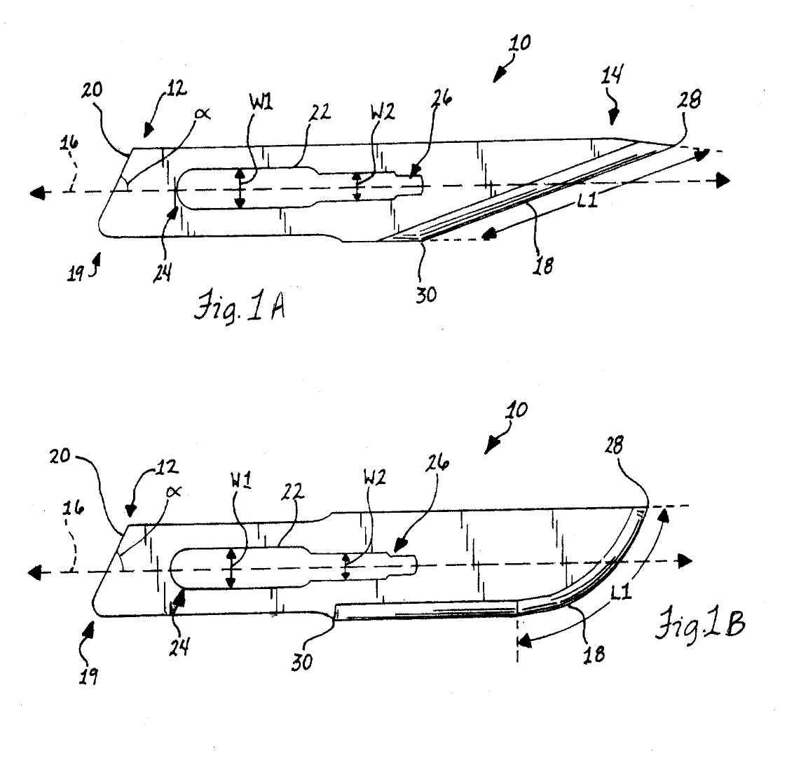

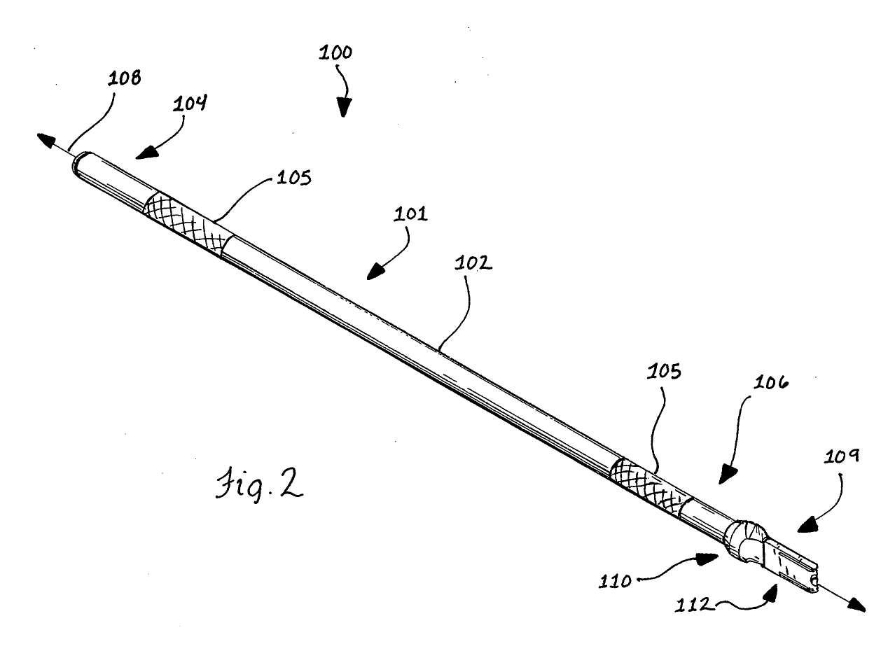

As shown in FIGS. 2-5, an annulus cutting tool 100 having a handle 101 is provided which is configured for the attachment of two surgical blades in the same orientation. The cutting tool 100 is of particular utility for forming substantially parallel sides of predetermined length of an opening in the wall of the annulus through which an artificial disc device can be inserted. Artificial disc devices that can be inserted through the openings made in the annulus with the tools herein can include those disclosed in co-pending applications Ser. Nos. 10/282,620 (now U.S. Pat. No. 7,001,433), 10/692,468, 10/971,734, 11/184,625, and 60/822,027, commonly assigned to the assignee herein and which are incorporated by reference as if reproduced in their entirety herein. After the sides are cut, the tool and blades are withdrawn from the annulus wall in which the parallel incisions were made, rotated ninety degrees, and the tool and blades are advanced into the annulus wall again to form parallel incisions that interconnect the previously cut parallel incisions for forming a window opening in the annulus wall having four sides through which the artificial disc device is to be inserted in the nuclear space. If the distance at which the blades are spaced from each other when mounted to the tool corresponds to the length of the incision those blades are preset to cut, then the opening will have four equal length sides and be square shaped. Manifestly, various sizes, shapes, and configurations of openings in the annulus wall can be formed with the tools described herein. For instance, the cutting tool herein is used to form openings having a rectangular shape, a substantially curved perimeter, a circular perimeter, or otherwise curved perimeter. The cutting tool herein may also be used to form openings that do not have a completely closed perimeter, such as to leave a flap of the cut annulus.

The cutting tool handle 101 includes an elongate shaft portion 102 with a proximal end portion 104 and a distal end portion 106 with a longitudinal axis 108. The distal end portion 106 of the elongate shaft portion 102 includes a mounting portion 109 for cooperating with the mounting portion 19 of the surgical blades. In the illustrated form, the mounting portion 109 includes a blade locator portion 110 for orientating the surgical blades relative to the elongate shaft portion 102 and a blade holder portion 112 for attachment of the surgical blades to the cutting tool elongate shaft portion 102.

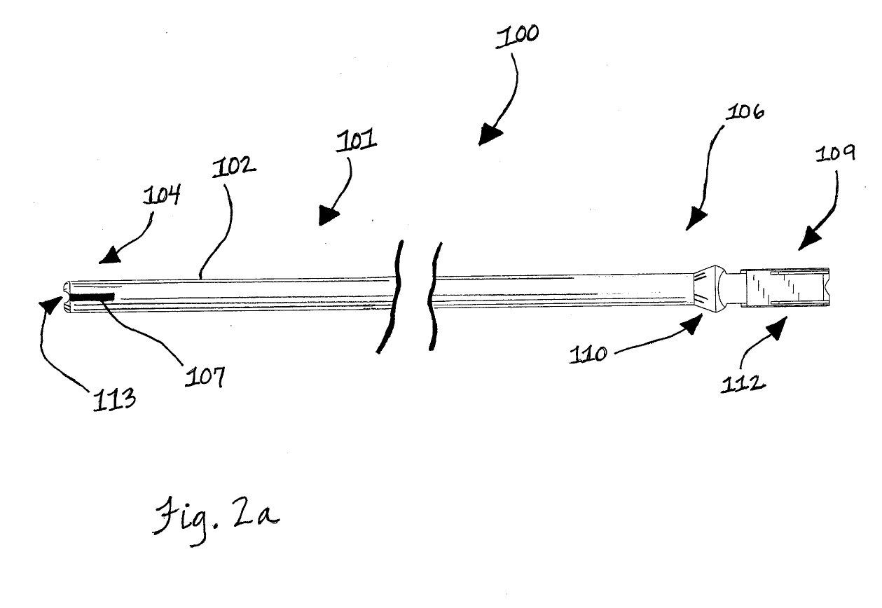

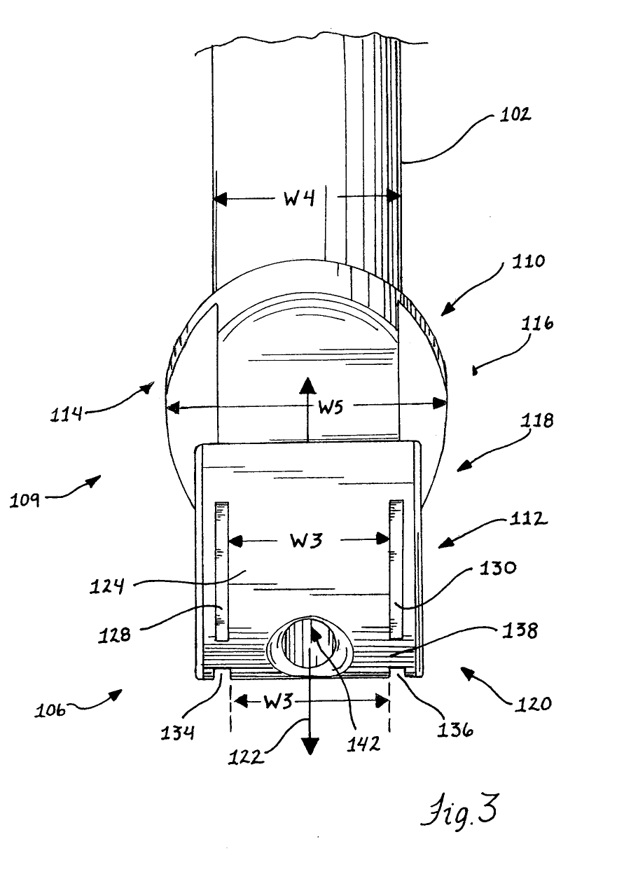

As shown in FIG. 3, the blade locator portion 110 is adjacent to and enlarged relative to the tool elongate shaft portion 102 and preferably includes indentations 114 and 116, with the orientation of locating surfaces in the indentations determining the orientation of the surgical blade. The reduced size blade holder portion 112 is at the distal end 106 so that the enlarged locator portion 110 is between the elongate shaft portion 102 and the blade holder portion 112. As best seen in FIG. 4, the enlarged blade locator portion 110 has a frustoconical portion 110a tapering away from the adjacent cylindrical shaft portion 102 to an enlarged cylindrical portion 110b in which the locating indentations 114 and 116 are formed on either side thereof and from which the blade holder portion 112 projects. The blade holder portion 112 has a generally rectangular, thin bar shaped configuration projecting from the enlarged blade locator portion 110 centrally aligned along the tool axis 108 via an arcuate transition section 111.

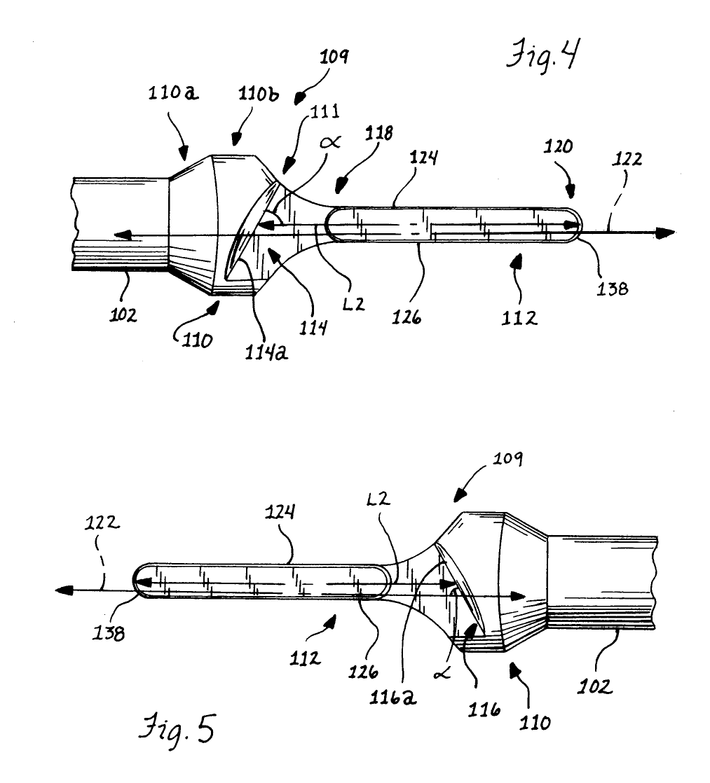

More particularly, indentation 114 can be better seen in FIG. 4. Rear locating surface 114a of the indentation 114 is oriented to form an acute angle α with a line parallel to the longitudinal axis 122 of blade holder portion 112. The indentation 116 can be better seen in FIG. 5. Rear locating surface 116a of the indentation 116 also forms an acute angle α with a line parallel to the longitudinal axis 122 of the blade holder portion 112. Because the locating surfaces 114a and 116a are configured to form the same acute angle α, the locating surfaces 114a and 116a provide the same orientation for both surgical blades 10 located thereagainst.

The mounting portion 109 of the elongate shaft portion 102 also includes a blade holder portion 112. As best seen in FIG. 4, blade holder portion 112 has a proximal end 118 and a distal end 120 oriented along a longitudinal axis 122. The blade holder portion 112 includes a first surface 124 and a second surface 126 opposite the first surface 124. Referring again to FIG. 3, the first surface 124 of the blade holder portion 112 includes linear recesses or elongate grooves 128 and 130, with the elongate grooves 128 and 130 being separated by a width W3 transverse to the longitudinal axis 122 of the blade holder portion 112. The second surface 126 of the blade holder portion 112 includes elongate grooves 134 and 136, with the grooves 134 and 136 being separated by the same width W3 as the grooves 128 and 130 transverse to the longitudinal axis 122 of the blade holder portion 112 so that grooves 128 and 134 are aligned with each other, and grooves 130 and 136 are aligned with each other on the blade holder portion 112 of the tool. The elongate grooves 128, 130, 134, and 136 extend parallel to the longitudinal axis 122 of the blade holder portion 112.

The width W3 between the elongate grooves may be varied in different embodiments of the cutting tool handle 101 depending on the desired predetermined distance between incisions. Surgeons would then have a variety of cutting tools to choose between based on the width of the opening needed in the annulus wall in a particular situation. The width W3 also corresponds to the length of the stop surface 138 extending between the blades 10 as described more fully hereinafter.

As shown in FIG. 3, the elongate shaft portion 102 generally has a diameter or width W4 that is smaller than the width W5 of the enlarged blade locator portion 110. The width W5 of the blade locator portion 110 is generally dictated by the width W3 between the elongated grooves. As the width W3 increases, the width W5 of the blade locator portion 110 increases correspondingly. If the width W4 of the elongate shaft portion 102 also increases as the width W5 increases, the width of the elongate shaft portion 102 may become undesirably wide and may reduce the comfortable use by the surgeon. Therefore, it is preferable to keep a predetermined width W4 for elongate shaft portion 102 to a minimum and generally smaller width than that of the locator portion W5 that is comfortable to the user.

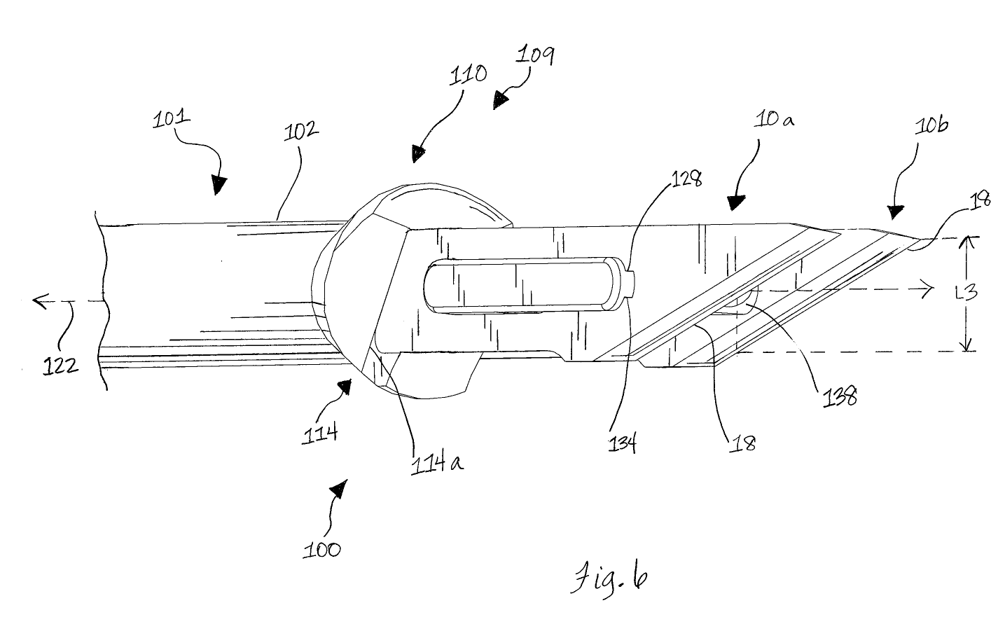

As shown in FIG. 6, two surgical blades 10 are mounted to the mounting portion 109 in the same orientation. A first surgical blade 10a is mounted to the mounting portion 109 by placing the larger width proximal end 24 of the surgical blade slot 22 about the blade holder portion 112 such that, when shifted back toward the blade locator portion 110, the slot edges abut the narrower distal end portion 26 of the elongate slot 22 and slide in the elongate grooves 128 and 134. The slot edges continue to slide in the retaining grooves 128 and 134 until the rear, inclined abutment surface 20 of the surgical blade 10a comes into close abutment with the inclined locating surface 114a to hold the surgical blade 10a on the cutting tool mounting portion 109. A second surgical blade 10b is mounted to the mounting portion 109 by placing the larger width proximal end 24 of the surgical blade slot 22 about the blade holder portion 112 such that, when shifted back toward the blade locator portion 110, the slot edges abut the narrower distal end portion 26 of the elongate slot 22 slide in the elongate grooves 130 and 136. The slot edges continue to slide in the retaining grooves 130 and 136 until the rear, inclined abutment surface 20 of the surgical blade 10b comes into close abutment with the inclined locating surface 116a (best seen in FIG. 5) to hold the surgical blade 10b on the cutting tool mounting portion 109. The surgical blades 10a, 10b and blade holder portion 112 are coupled in a releasable fixed orientation such that a surgeon may apply force to the cutting tool handle 101 for cutting with the blades 10a, 10b without the surgical blades separating therefrom since the cutting force will tend to push the blades 10a, 10b more tightly against the locating surfaces 114a and 116a with the blade holder portion 112 also being pushed more tightly into the narrow distal end portion 26 of the blade slot 22.

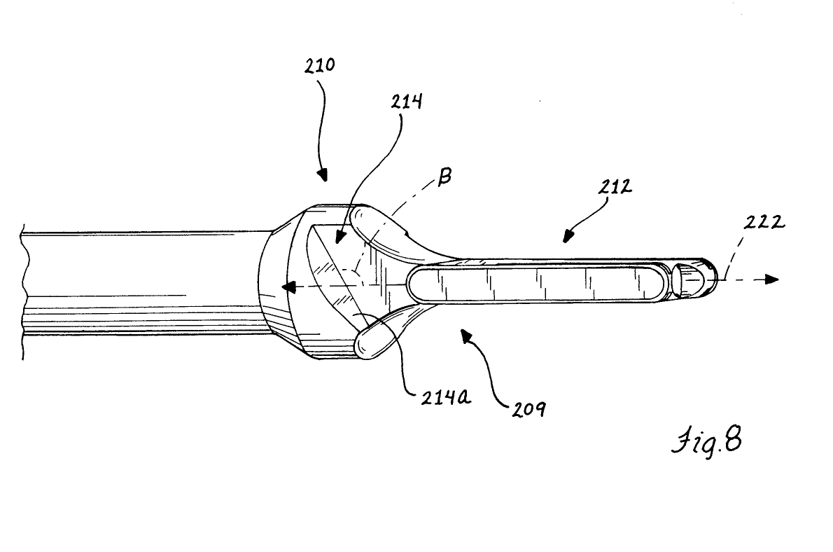

It should be noted that the configuration of the indentations 114 and 116 may be reversed such that indentations 114 and 116 form obtuse angles β, such as that seen in FIG. 8 and discussed in more detail below, with a line parallel to the longitudinal axis 122 of the blade holder 112 such that the indentations 114 and 116 provide a substantially opposite orientation from that illustrated in FIG. 6.

As best shown in FIG. 3, the distal end 120 of the blade holder portion includes a stop surface 138 at a predetermined position relative to the length of the inclined cutting edges 18 of the blades 10 so as to substantially define the length of the incision in the annulus wall made therewith. The stop surface 138 extends for a sufficient distance transverse to the cutting edges 18, such as extending between the elongate grooves 128, 130, 134, and 136 for a distance W3 as illustrated so that the tool bar portion 112 cannot be pushed into the incisions, stopping continued cutting of the annulus wall with the blade edges 18. The stop surface 138 also may extend for a short distance beyond the blades 10 and their retaining grooves 128, 130, 134, and 136, although it is primarily the length of the stop surface 138 extending between the blades 10 that serves to act as the stop against continued cutting.

As best shown in FIGS. 4 and 5, the stop surface 138 is spaced from indentations 114 and 116 by a fixed length L2 parallel to the longitudinal axis 122 of the blade holder portion 112. Since the stop surface 138 will be at a predetermined position recessed back from the blade distal tip 28 of the cutting blades 10, this will allow the length of the incision made by the inclined cutting edges 18 thereof to be known depending on the amount of inclination or declination of the cutting edges 18 and their length relative to the location of the stop surface 138. As illustrated in FIG. 6, the stop surface 138 is at an intermediate position in the longitudinal direction along axis 122 relative to the blade edges 18 extending forwardly and rearwardly thereof in the longitudinal direction. Accordingly, the stop surface 138 will keep the portions of the blade edges 18 extending rearwardly therefrom from cutting into the annulus wall, thus defining the length of the incision as being approximately the distance L3 from the stop surface to the distal edge 28 of the blades in a direction orthogonal to the longitudinal axis.

During surgery, the stop surface 138 is the rounded, free end of the bar holder portion 112 and is sized so as to be operable to contact the annulus and limit the size of the incision made in the annulus made by the blades 10 to a predetermined length. For example, the stop surface 138 contacts the annulus to limit the length of the incision by limiting the portion of the length L1 of the cutting edge 18 that pierces and cuts the annulus.

Accordingly, the cutting tool 100 allows a surgeon to create parallel incisions of identical length in a controlled manner. Once the surgeon has identified the appropriate location in the annulus for the incisions, the surgeon applies force to the cutting tool handle 101 to direct the surgical blades into engagement with the annulus for cutting the wall thereof. The surgeon may continue to apply force until the stop surface 138 of the cutting tool handle 101 contacts the annulus. The stop surface 138 extending transversely to the thin cutting edges 18 of the blades 10, and preferably orthogonal thereto, for a distance sufficient that the stop surface 138 cannot be fit into the incisions made by the blades 10 without damaging the annulus wall, as by tearing thereof. Accordingly, the transverse stop surface 138 keeps the surgical blades from advancing further into the annulus wall once the stop surface 138 is engaged therewith, thus limiting the size of the incisions to a predetermined length, as explained above.

To complete cutting the opening in the annulus, the surgeon removes the cutting tool blades 10 from the annulus, rotates the cutting tool 100 by 90 degrees in a clockwise or counterclockwise direction, and then makes a second set of parallel incisions of identical length in the annulus.

As shown in FIG. 2, the elongate shaft portion 102 may further include one or more gripping surfaces 105. For example, the gripping surface may be grooves, ridges, or corrugations on the elongate shaft portion 102 to enhance gripping ability. Preferably, the gripping surface is some kind of texturization or roughening to the otherwise smooth surface of the elongate shaft portion 102 which allows cleaning and sterilization of the cutting tool handle 101, such as by autoclaving. In one aspect, one or more gripping surfaces may be located anywhere on the elongate shaft portion 102, such as at the proximal end portion 104 and/or at the distal end portion 106 of the elongate shaft portion 102. In another aspect, a substantial portion of the elongate shaft portion 102 may have a gripping surface.

In another aspect, the elongate shaft portion 102 may further include indicia so that the surgeon can easily identify the orientation of the surgical blades. Preferably, the indicia are located at the proximal end of the elongate shaft portion 102 so that the surgeon can identify the orientation of the blades when the blades have been inserted into the annulus wall. As shown in FIG. 2A, for example, the indicia may include any markings known in the art, such as etchings 107, and/or a machined groove 113 at the proximal end 104 of elongate shaft portion 102. The etchings 107 and/or machined groove 113 are oriented that a line extending therefrom toward the mounting portion 109 will extend between the parallel blades 10 thereto so that the blades 10 are on either side of the reference line. In this manner, the surgeon will know that the blades are in their generally vertical orientation, as shown in FIG. 6, for example.

Because proper identification of the point of incision and precise cutting of the annulus is important for disc replacement surgery, the cutting tools described herein may be cannulated and used in conjunction with a guide wire. Use of a guide wire is not required for proper functioning of the cutting tools described herein and depends on the preference of the surgeon. As shown in FIG. 3, the elongate shaft portion 102, the blade locator portion 110, and blade holder portion 112 may include a central longitudinal throughbore 142 extending from the proximal end 104 of the elongate shaft portion 102 to the stop surface 138 of the blade holder portion 112. If the surgeon desires to use a guide wire with any of the cutting tools described herein, the surgeon may insert a guide wire into a patient's annulus at the desired location for incision and drive the guide wire to a desired depth until secure. Each segment of the procedure may be guided by fluoroscopic or other imaging to assure accurate and safe placement of the guide wire and to prevent driving the guide wire through and beyond the walls of the annulus.

The guide wires used in conjunction with the cutting tools of the invention are preferred to have a self-cutting and self-tapping thread; however, the thread type and insertion means may vary by surgeon preference. Alternatively, the guide wire may have a non-threaded sharpened end for advancement into the annulus and is preferably constructed of biocompatible metals or alloys such as stainless steel, titanium, or nitinol. Once the guide wire is secured at the desired position in the annulus, the throughbore 142 at the distal end 120 of the blade holder portion is guided onto the free end of the guide wire and advanced along the guide wire towards the annulus. The surgeon then makes the predetermined incision(s) in the annulus. The surgeon may then remove the cutting instrument from the guide wire. The surgeon may then remove the guide wire from annulus or remove the excised portion of the annulus which may still be attached to the guide wire.

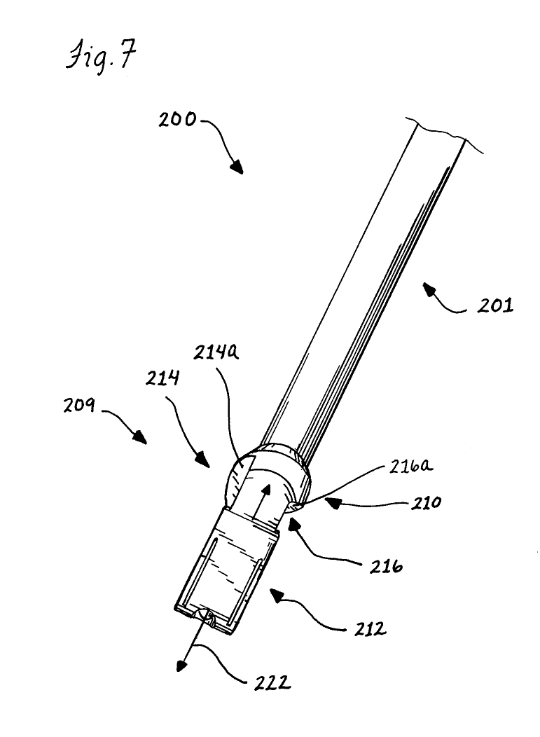

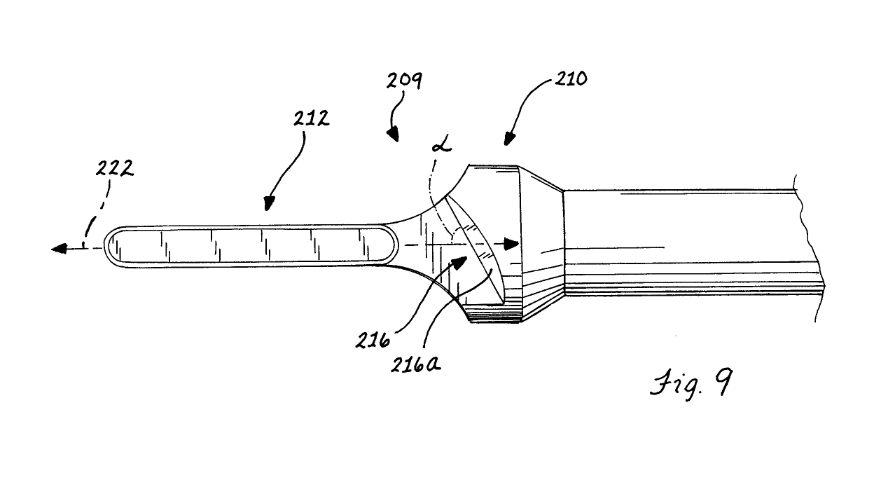

Referring to FIGS. 7-10, an alternative cutting tool 200 is shown. Cutting tool handle 201 has substantially the same structure as cutting tool handle 101 but has an alternative configuration of the blade mounting portion 209 over blade mounting portion 109. As shown in FIG. 7, the blade locator portion 210 includes indentations 214 and 216 including rear locating surfaces 214a and 216a thereof, the orientation of which determines the orientation of the surgical blade. Indentation 214 is best seen in FIG. 8. Locating surface 214a forms an obtuse angle β with a line parallel to the longitudinal axis 222 of the blade holder portion 212. Indentation 216 can be better seen in FIG. 9. Locating surface 216a forms an acute angle α with a line parallel to the longitudinal axis 222 of the blade holder portion 212. Manifestly, the configuration of the indentations may be reversed such that locating surface 214a forms an acute angle α with a line parallel to the longitudinal axis 222 of the blade holder 212 while locating surface 216a forms an obtuse angle β with a line parallel to the longitudinal axis 222 of the blade holder portion 212.

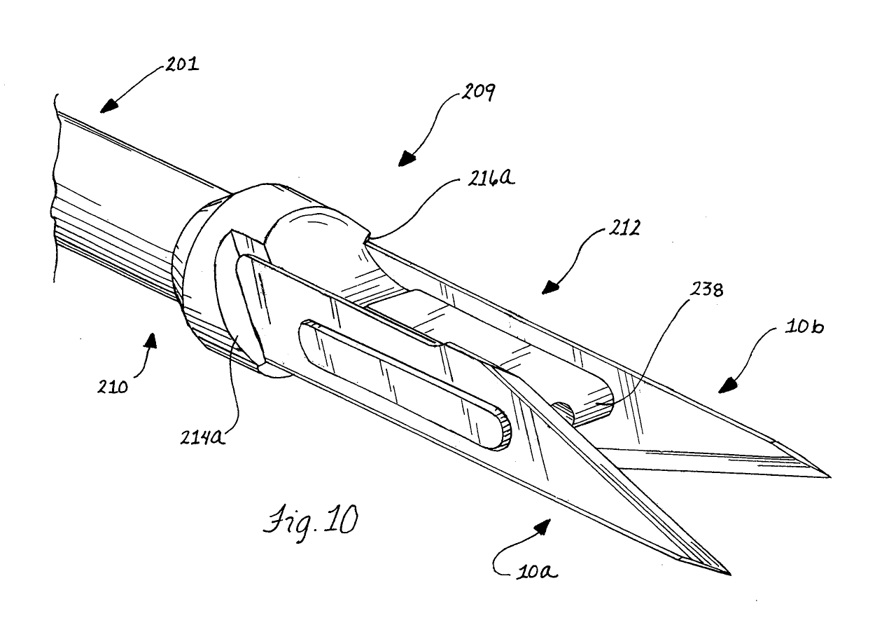

As shown in FIG. 10, a first surgical blade 10a and a second surgical blade 10b are mounted on the blade mounting portion 209 on the cutting tool handle 201. Because locating surface 214a forms an obtuse angle β with a line parallel to the longitudinal axis 222 (as shown in FIG. 8), the first surgical blade 10a will be positioned with a first orientation. Because locating surface 216a forms an acute angle α with a line parallel to the longitudinal axis 222 (as shown in FIG. 9), the second surgical blade 10b will be positioned with an opposing second orientation.

The cutting tool 200 also allows a surgeon to create parallel incisions in a controlled manner. Once the surgeon has identified the appropriate location in the annulus for the incisions, the surgeon applies force to cutting tool handle 201 sufficient to cut into the annulus with the reversely oriented blades 10a and 10b. Again, as explained above in reference to the cutting tool handle 101, the surgeon may apply force until the transversely extending stop surface 238 of the cutting tool handle 201 contacts the annulus wall. The stop surface 238 keeps the surgical blades 10a, 10b from continuing to advance into the annulus, thus limiting the length of the incisions, as explained above.

In addition to the method described above in reference to cutting tool 100, if the surgeon desires to cut an arcuate opening in the annulus, the surgeon may simply twist or rotate the cutting tool 200 by approximately 180 degrees in the annulus wall to form an arcuate incision so that the opening has a generally circular configuration or a substantially curved perimeter, for example.

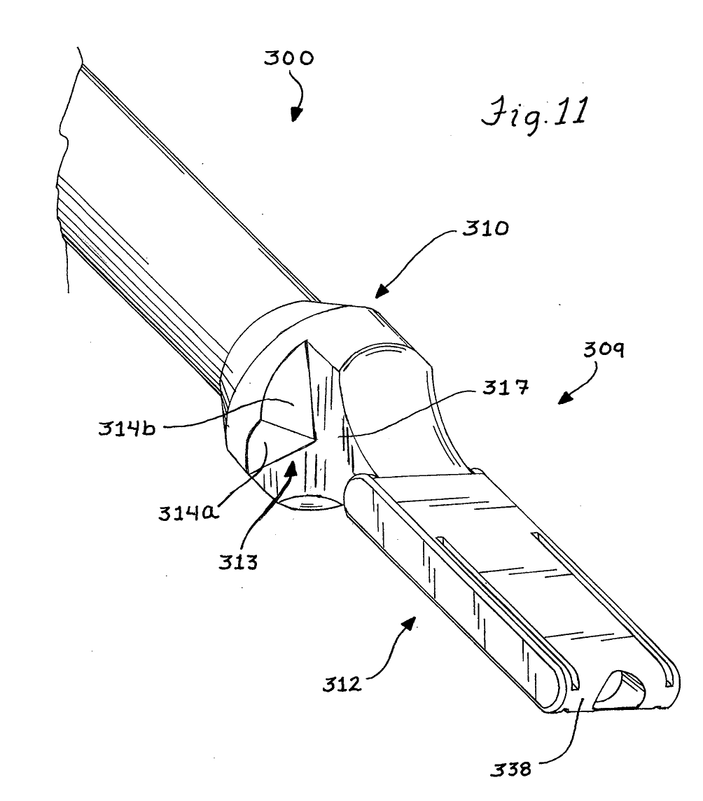

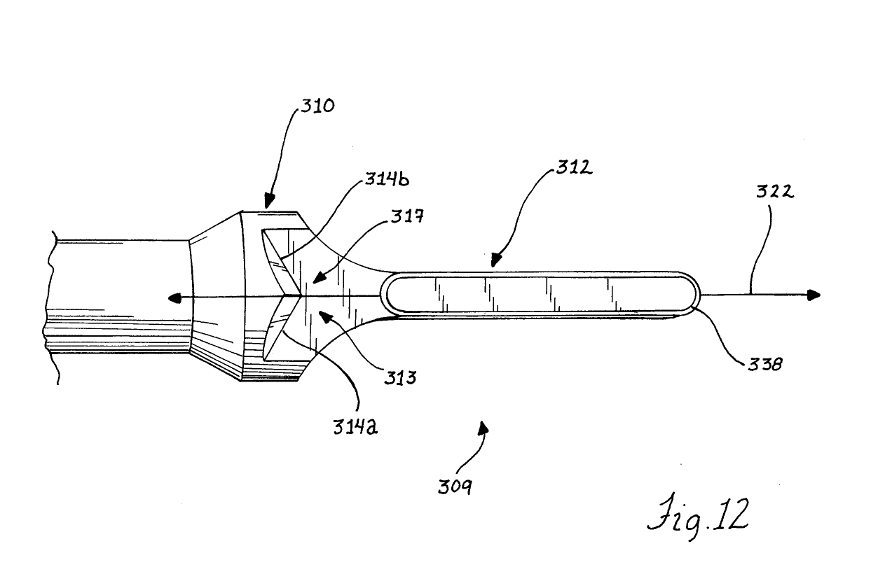

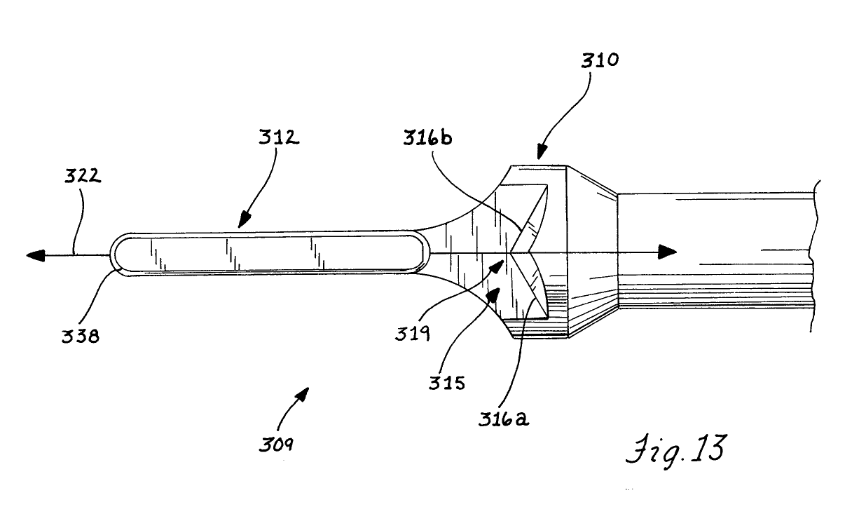

FIGS. 11-14 illustrate a cutting tool 300 with substantially the same structure as cutting tools 100 and 200 but with another alternative configuration of the blade mounting portion 309 over the previously described blade mounting portions 109 and 209. The blade mounting portion 309 also includes a blade locator portion 310 and a blade holder portion 312. As shown in FIGS. 11 and 12, the blade locator portion 310 includes an indentation 313 with surface portions 314a and 314b to form a V-shaped locating surface 317 and, as shown in FIG. 13, an indentation 315 with surface portions 316a and 316b to form a V-shaped locating surface 319. As shown in FIGS. 12 and 13, the V-shaped locating surfaces 317 and 319 allow the surgeon great flexibility in selecting the orientation of the surgical blades because each surgical blade may be mounted in either one of two orientations via the V-shaped locating surfaces. Therefore, a first surgical blade and a second surgical blade may be mounted in a same orientation or in opposing orientations.

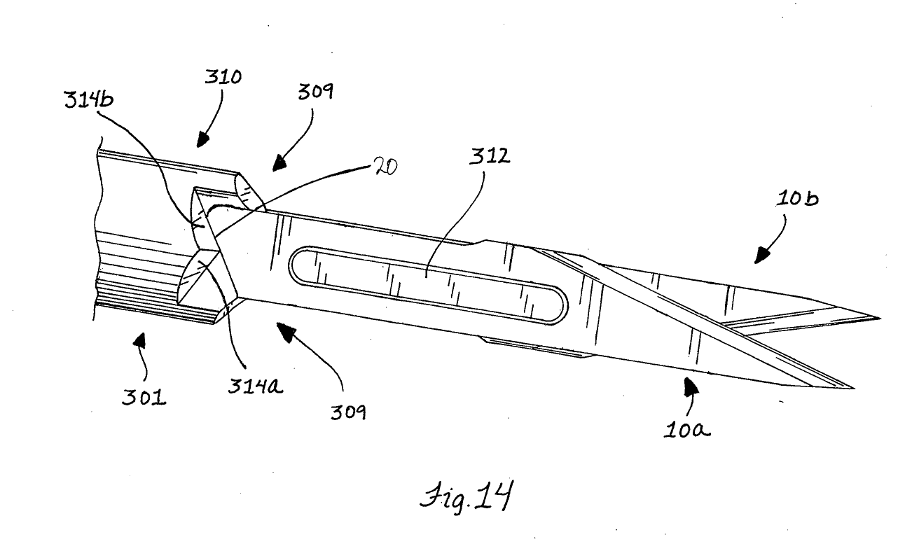

As shown in FIG. 14, a first surgical blade 10a and a second surgical blade 10b are located in opposing orientations on the blade mounting portion 309 of the cutting tool handle 301. The first surgical blade 10a is positioned in a first orientation with its rear abutment surface 20 engaged flush against inclined surface portion 314b with the blade abutment surface 20 also extending beyond the surface portion 314b and diverging away from surface portion 314a. To reversely locate the first blade 10a, the blade 10a can be turned 180 degrees about its axis 16 (shown in FIG. 1) so that the rear abutment surface 20 can be engaged flush with the locating surface portion 314a. Again, in this instance only a portion of the blade abutment surface 20 engages against surface portion 314a with the remainder thereof extending beyond the surface portion 314a and diverging away from surface portion 314b. The same will hold true for the other blade 10b and locating surface portions 316a and 316b (not shown in FIG. 14) so that the blade 10 can be mated against either one of these surface portions 316a and 316b to provide it with two different orientations on the tool 300 that the surgeon can select. The blade 10 can be turned 180 degrees about its axis 16 so that the rear abutment surface 20 can be engaged flush with either of locating surface portions 316a and 316b. As illustrated in FIG. 14, the second surgical blade 10b is positioned in a reverse orientation from the first blade 10a. It should be noted that the surgical blades could also be mounted in the same orientation, if desired.

The cutting tool 300 allows a surgeon great flexibility to create parallel incisions in a controlled manner. Once the surgeon has identified the appropriate location in the annulus for the incisions, the surgeon applies force to the cutting tool handle 301 sufficient so that the surgical blades cut into the annulus wall. Again, as explained above in reference to the cutting tool handle 101, the stop surface 338 of the cutting tool handle 301 will contact the annulus wall and keep the surgical blades from advancing into the annulus beyond a predetermined distance, thus limiting the length of the incisions, as explained above.

If the surgical blades are positioned in the same orientation on the cutting tool handle 301, the cutting tool handle 301 may be used as described above for the cutting tool handle 101, where the surgical blades are inserted into the annulus to create two parallel incisions of substantially identical length, withdrawing the surgical blades from the annulus wall, rotating the cutting tool handle 301 by ninety degrees in a clockwise or counterclockwise direction, and then creating a second set of parallel incisions of identical length in the annulus.

If the surgical blades are positioned in opposing or reverse orientations, the cutting tool handle 301 may be used as described above for the cutting tool handle 201, where the surgical blades are inserted into the annulus to create two parallel incisions and then the surgeon may simply twist or rotate the cutting tool handle 301 to form an arcuate or substantially circular incision in the annulus.

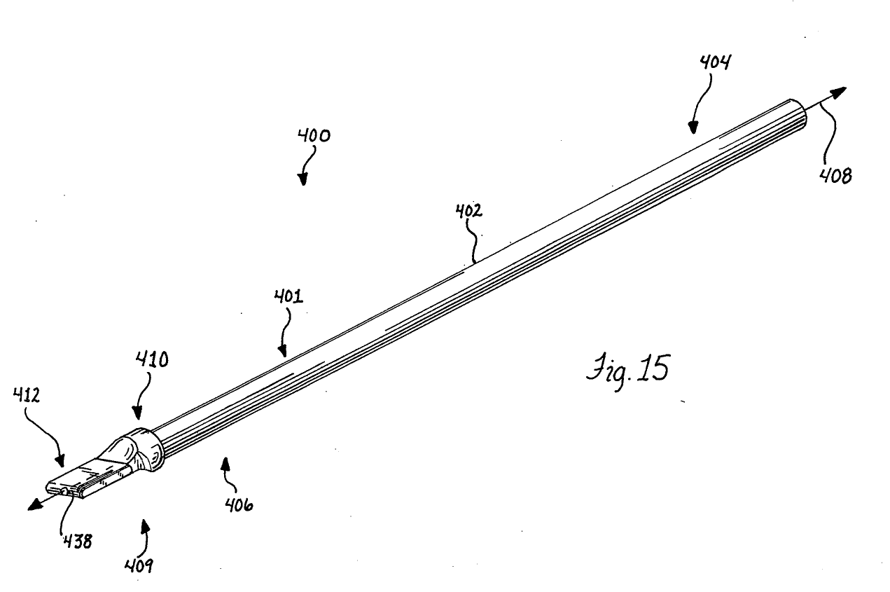

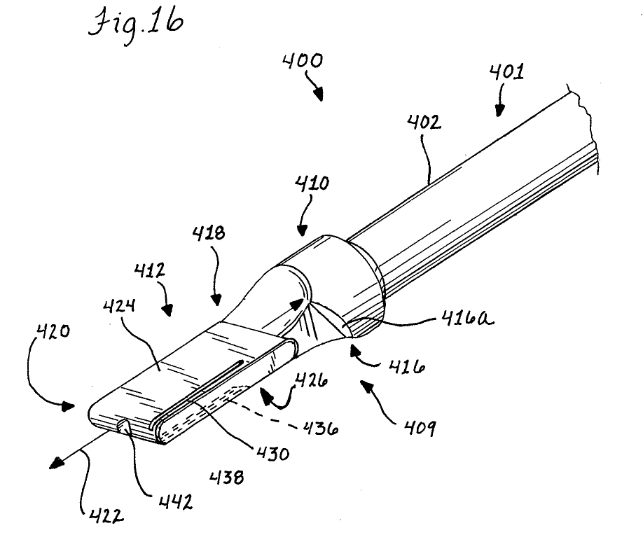

FIGS. 15-16 illustrate a cutting tool 400 having substantially the same structure as cutting tools 100, 200, and 300 but with an alternative configuration of the blade mounting portion 409. As shown in FIG. 15, the mounting portion 409 of the cutting tool 400 is configured for mounting only a single surgical blade 10. The other blade cutting tools 100, 200, and 300 may be used with only a single blade 10 mounted on one side or the other of their blade mounting portions; however, tool 400 is specifically adapted for this purpose as it only includes one set of retaining grooves 430, 436 as described hereafter.

The cutting tool handle 401 includes an elongate shaft portion 402 with a proximal end portion 404 and a distal end portion 406 with a central, longitudinal axis 408. The distal end portion 406 of the elongate shaft portion 402 includes a blade mounting portion 409 having a blade locator portion 410 and a blade holder portion 412 with a stop surface 438. As shown in FIG. 16, the blade locator portion 410 includes a single indentation 416 including a single rear locating surface 416a thereof, the orientation of which determines the orientation of a single surgical blade, as described above.

As shown in FIG. 16, the blade holder portion 412 has a proximal end portion 418 and a distal end portion 420 and a longitudinal axis 422 extending therebetween coaxial with the axis 408 (shown in FIG. 15). The blade holder portion 412 includes a first surface 424 and a second surface 426 opposite of the first surface 424. The first surface 424 of the blade holder portion 412 includes linear recess or elongate retaining groove 430. The second surface 426 of the blade holder portion 412 includes linear recess or elongate retaining groove 436 aligned with retaining groove 430. The elongate grooves 430 and 436 extend generally parallel to the longitudinal axis 422 of the blade holder portion 412 and one side thereby offset from the tool axis 408.

Only a single surgical blade can be mounted on the cutting tool handle 401. A surgical blade with at least a partially curved cutting edge, such as a blade similar to that depicted in FIG. 1B, may be used with the cutting tool handle 401 to create an arcuate incision in the annulus, such as for forming a generally circular opening in the annulus wall.

It is preferable for the cutting tool handle 401 to be cannulated as illustrated for use in conjunction with a guide wire. Use of a guide wire, as described in detail above, is particularly useful when creating arcuate or circular incisions in the annulus. As shown in FIG. 16, the cutting tool handle 401 includes a longitudinal throughbore 442 through which a guide wire is received. Once the guide wire is secured at the predetermined position in the annulus wall, the opening to the longitudinal throughbore 442 at the distal end portion 420 of the blade holder portion 412 is guided onto the free end of the guide wire. The tool 400 is then advanced along the guide wire towards the annulus to bring the blade 10 into engagement therewith. The surgeon then makes plunge cut with the blade 10 into the annulus wall until the stop surface engages the annulus wall. Thereafter, the surgeon can make a controlled circular incision by turning the cutting tool handle 401 about the guide wire. The surgeon may then withdraw the cutting instrument off from the guide wire. Then, the guide wire is removed from annulus, if necessary, or from any excised portion of the annulus which may still be attached to the guide wire.

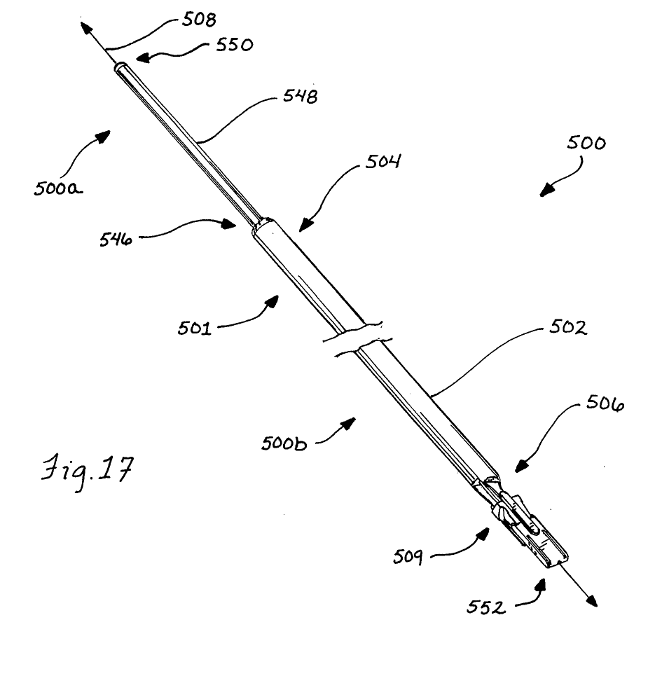

FIGS. 17-20 illustrate an alternative embodiment of a cutting tool 500 capable of having more than two and up to four cutting blades 10 attached thereto. As shown in FIG. 17, the annulus cutting tool 500 has a handle or shaft assembly 501 that includes two shaft members, an inner shaft member 500a for mounting one or two surgical blades and an outer shaft member 500b for mounting one or two surgical blades. The shaft members 500a and 500b may be used alone or in combination to create incisions of the desired length. When used in combination as an assembly, the surgical blade(s) mounted on the inner shaft 500a are generally positioned perpendicular to the surgical blade(s) mounted on the outer shaft member 500b.

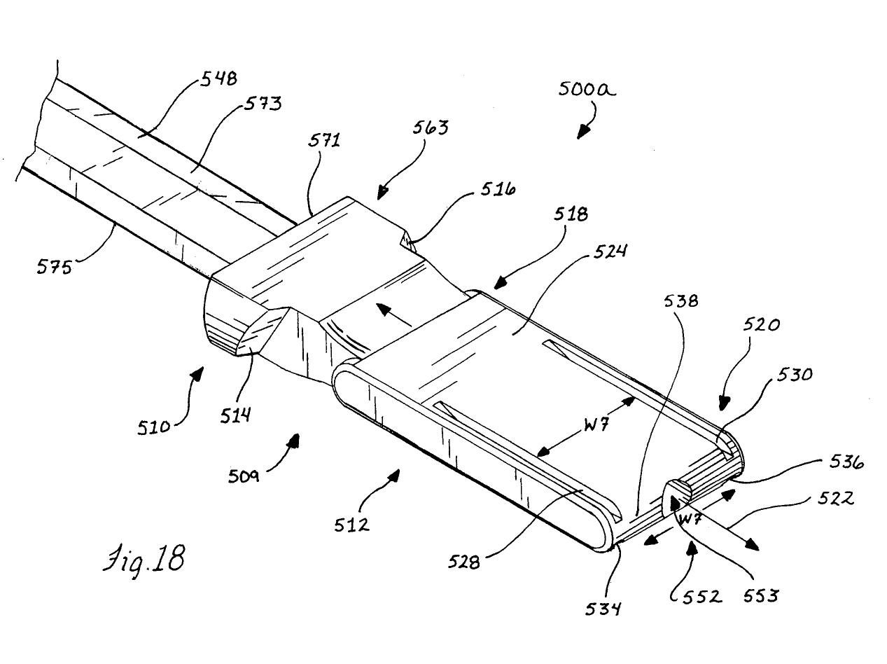

As shown in FIGS. 17 and 18, the inner shaft member 500a includes an inner, elongate rod portion 548 having a proximal end 550 and a distal end 552, and a blade mounting portion 509. As shown in FIG. 18, the blade mounting portion 509 has a blade locator portion 510 and a blade holder portion 512 adjacent the distal end 552. As shown in FIG. 17, the shaft portion 548 of the inner shaft member 500a has an outer diameter sized to permit the elongate rod portion 548 to easily reciprocate within a throughbore 546 of the outer shaft member 500b.

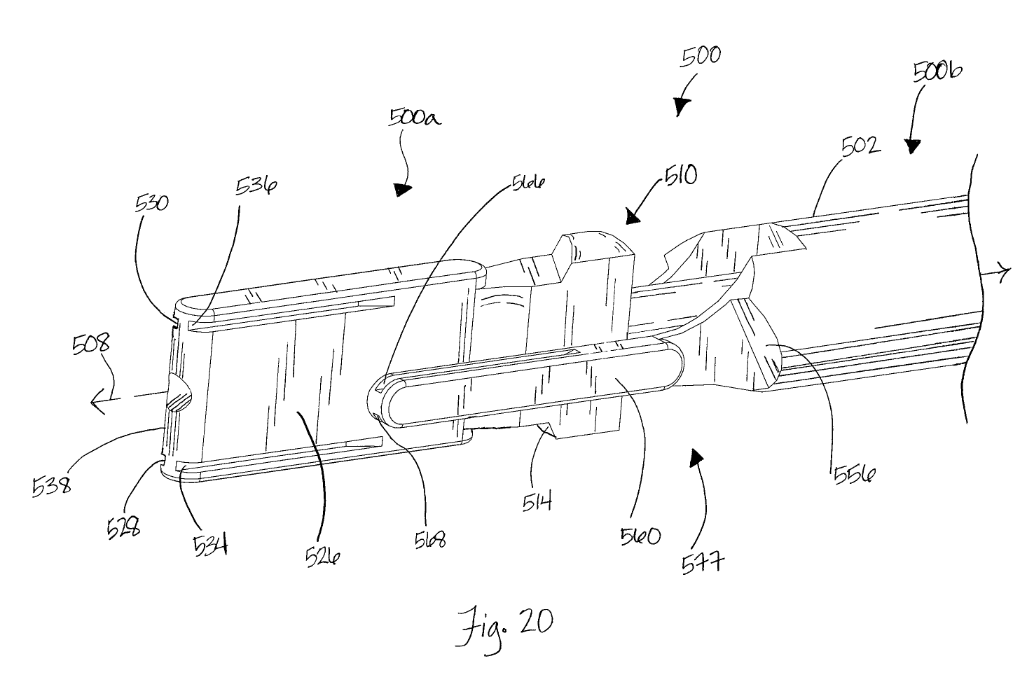

As shown in FIG. 18, the blade locator portion 510 includes indentations 514 and 516, the orientations of which determine the orientation of the surgical blades. The blade holder portion 512 of the inner shaft member 500a has a proximal end portion 518 and a distal end portion 520 oriented about a longitudinal axis 522. The distal end portion 520 of the blade holder portion 512 includes a stop surface 538. The blade holder portion 512 includes a first surface 524 and a second surface 526 opposite the first surface (shown in FIG. 20). The first surface 524 of the blade holder portion 512 includes linear recesses or elongate grooves 528 and 530 with the elongate grooves 528 and 530 being separated by a width W7 transverse to the longitudinal axis 522 of the blade holder portion 512. The second surface of the blade portion 512 includes elongate grooves 534 and 536, with the elongate grooves 534 and 536 being separated by a distance W7 transverse to the longitudinal axis 522 of the blade holder portion 512. The elongate grooves 528, 530, 534, and 536 are parallel to the longitudinal axis 522 of the blade holder portion 512.

As shown in FIG. 18, the inner shaft member 500a may include a central longitudinal throughbore 553 extending from the proximal end 504 of the elongate shaft portion 502 to the stop surface 538 of the blade holder portion 512. Therefore, the inner shaft member 500a may be used in conjunction with a guide wire as described in detail above in reference to cutting tool 100.

As shown in FIG. 17, the outer shaft member 500b includes an elongate hollow shaft or sheath portion 502 having a proximal end 504 and a distal end 506 oriented along a longitudinal axis 508. The sheath portion 502 includes a longitudinal throughbore 546 in which the elongate rod portion 548 of the inner shaft member 500a is received.

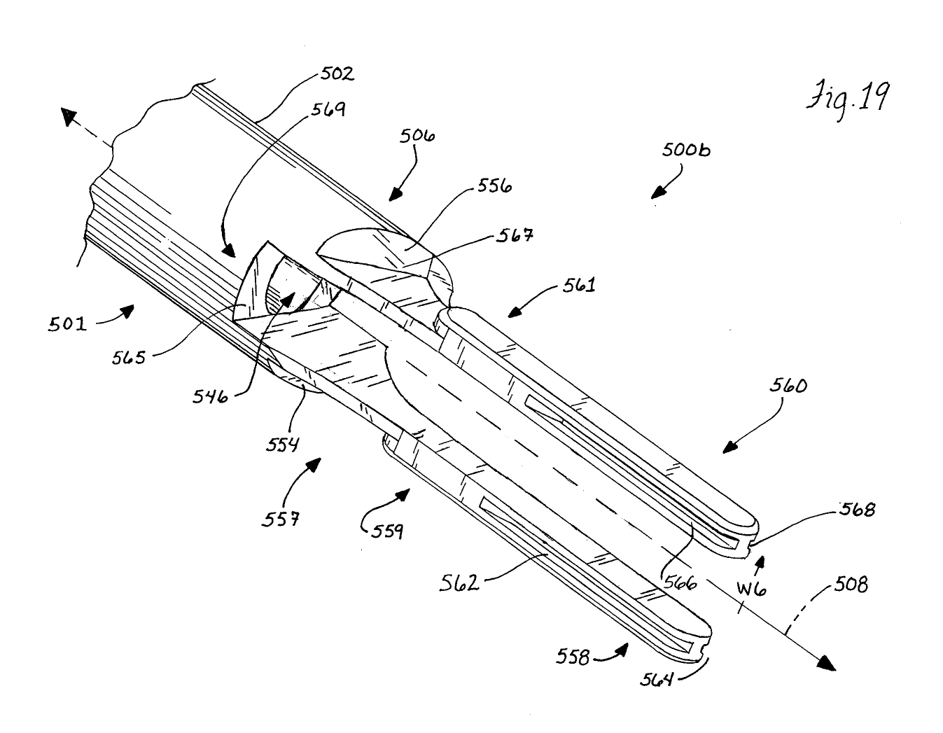

As shown in FIG. 19, the distal end 506 of the sheath portion 502 of the outer shaft member 500b includes a split mounting portion 557 including respective indentations 554 and 556 and blade holder projecting segments 558 and 560 formed in the split portions 559 and 561 thereof. The blade holder segment 558 includes aligned linear recesses or elongate retaining grooves 562 and 564 and the blade holder segment 560 includes aligned linear recesses or elongate retaining grooves 566 and 568. The elongate grooves 562 and 564 are separated from elongate grooves 566 and 568 by a width W6.

Although it is possible to use the members 500a and 500b independently of each other to cut an opening in the annulus wall, it is preferred that members 500a and 500b be used as an assembly 501 so that the opening is formed in a single cutting operation. The blades 10 can be mounted to the members 500a and 500b either before or after the elongate rod portion 548 is inserted into the hollow sheath portion 502.

Turning to more of the details and referring again to FIG. 18, at least one surgical blade may be mounted to the mounting portion 509 as described in detail above in reference to mounting portion 109. Referring again to FIG. 19, two surgical blades may be mounted on holder projecting segments 558 and 560 of the outer shaft member 500b generally as described above. A first surgical blade may be mounted on the outer shaft member 500b by placing the larger width proximal end 24 of the surgical blade slot 22 about the holder projecting segment 558 such that, when shifted back toward blade locator portion, the elongate slot distal end 26 of a first surgical blade is received in elongate retaining grooves 562 and 564. The slot edges continue to slide in the elongate retaining grooves 562 and 564 until the rear, inclined abutment surface 20 of the surgical blade 10 comes into close abutment with indentation 554 to secure the surgical blade 10 to the outer shaft member 500b. A second surgical blade 10 is mounted on outer shaft member 500b by placing the larger width proximal end 24 of the surgical blade 10 about the holder projecting segment 560 such that, when shifted back toward the blade locator segment 560, the elongate slot distal end 26 of a second surgical blade 10 is received in the elongate retaining grooves 566 and 568. The slot edges continue to slide in the elongate retaining grooves 566 and 568 until the abutment surface 20 of the surgical blade 10 comes into close abutment with indentation 556 to secure the surgical blade 10 to the holder projecting segment 560. The surgical blades 10 and holder projecting segments 558 and 560 are coupled in a releasable fixed orientation such that a surgeon may apply force to the cutting tool handle 501 for cutting with the blades without the surgical blades separating therefrom as described above.

As mentioned, the above described blade attachment can occur before or after the members 500a and 500b are assembled together. For assembly, the proximal end 550 of the elongate rod portion 548 of member 500a is inserted into the throughbore 546 of the outer shaft member. The split mounting portions 559 and 561 of member 500b have a transverse spacing therebetween that is greater than the thickness of the enlarged block-shaped locator portion 563 of member 500a in a direction orthogonal to the flat surfaces 524 and 526 thereof. Diametrically opposed, arcuate seating surfaces 565 and 567 are formed in the wall 569 of the sheath portion 502 of member 500b and recessed back from the spaced blade mounting segments 558 and 560. The rear abutment surface 571 of the block portion 563 has an arcuate configuration to substantially match that of the arcuate seating surfaces 565 and 567 of member 500b for flush engagement thereagainst. In addition, the elongate rod portion 548 preferably includes flats 573 and 575 and the interior surface of the cylindrical wall 569 includes flats (not shown) such that the rod portion 548 can only be inserted in the throughbore 546 if the blade holder portion 512 is oriented to fit between the split mounting portions 559 and 561 as shown in FIG. 20. In this regard, the elongate retaining grooves 528, 530, 534, and 536 of the inner shaft member 500a are positioned so that the blades held therein are oriented substantially perpendicularly to the blades held in elongate grooves 562 (not shown), 564 (not shown), 566, and 568 of the outer shaft member 500b when members 500a and 500b are coupled. Alternatively, the blades need not be mounted during this assembly as shown in FIG. 20. It should be noted that once the block portion is engaged against the seat surfaces 565 and 567, the members 500a and 500b may be secured to each other in this assembled position via a detent mechanism or other means for securing the first member to the second member.

With members 500a and 500b assembled prior to making any incisions in the annulus, the mounting portions 509 and 557 are combined to form a combined mounting portion or mounting assembly 577 for the tool 500 that can hold four surgical blades 10 so that four incisions may be made simultaneously in the annulus, such as a square or rectangular shaped incision, if desired.

It may be desired to mount a total of three surgical blades on the first and second cutting members 500a and 500b. For example, two surgical blades are mounted on the first cutting member 500a and one surgical blade is mounted on the second cutting member 500b. In another example, one surgical blade is mounted on the first cutting member 500a and two surgical blades are mounted on the second cutting member 500b. In this aspect, the three surgical blades are used to cut a three-sided incision in the annulus. When a three-sided incision is made in the annulus, the fourth, uncut side may form a flap portion that covers the three-sided opening of the annulus. Creating an annulus flap portion may be desirable because it may be possible to suture or stitch the annulus flap portion to allow for the annulus to heal such that it would be even more unlikely for an artificial disc to back out through the opening in the annulus.

Alternatively, although not pictured, instead of having both inner and outer shafts, the cutting tool handle 501 may have a single shaft with a proximal end portion and a distal end portion, with the distal end portion including both blade mounting portions and the two blade holder segments as described above. Blades mounted on the mounting portion would be perpendicular to the blades mounted on the blade holder segments, with the blades forming a box-shaped configuration. Locating the blade mounting portion and the blade holder segments on the same shaft provides a tool configured for cutting a box-shaped incision in the annulus wall. This tool could also be used to create a three-sided incision by mounting three blades on the tool, with the three-sided incision not having a completely closed perimeter so that a flap of the cut annulus wall remains after the cutting tool herein is used.

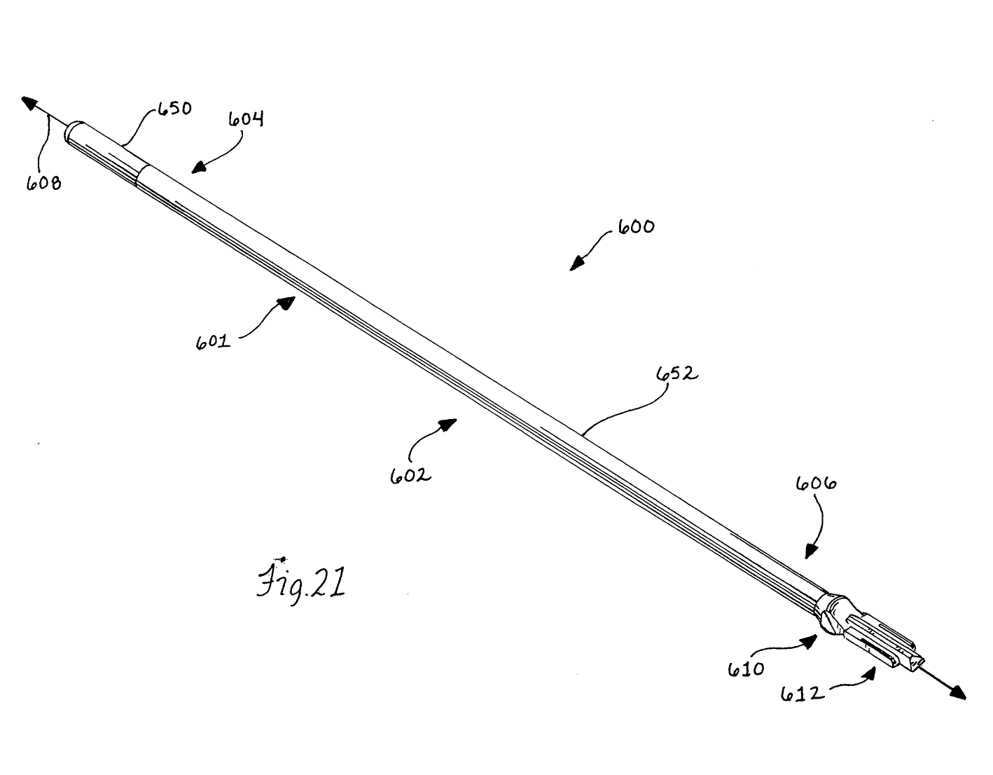

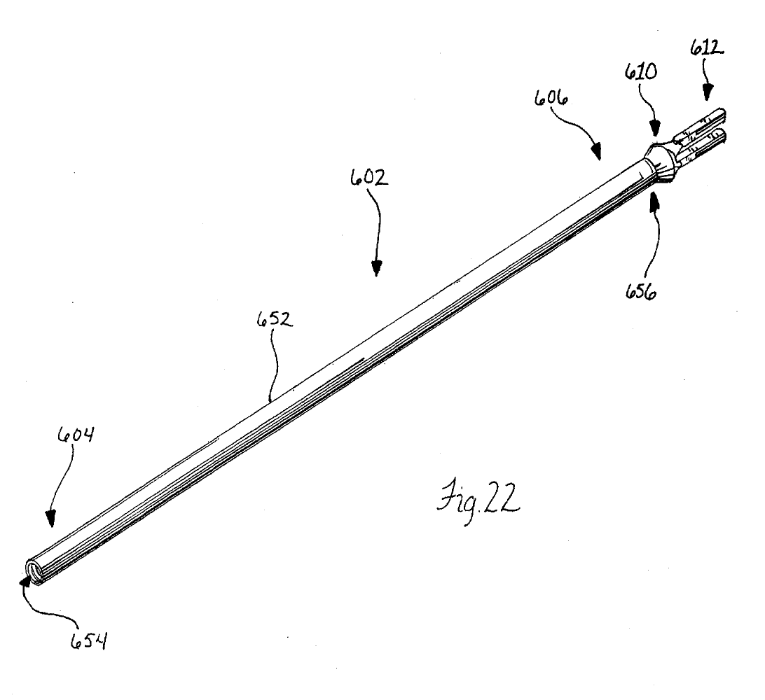

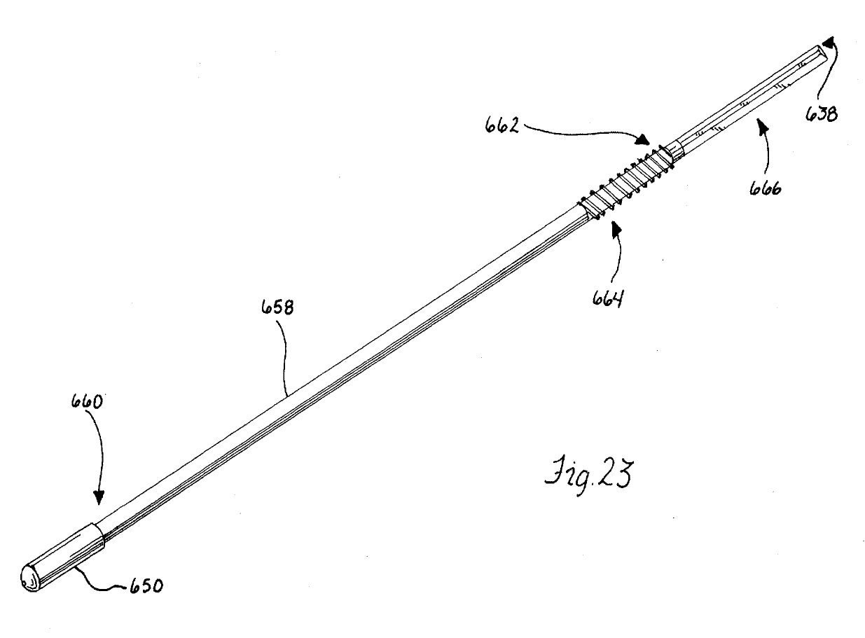

FIGS. 21-24 illustrate a cutting tool 600 with an adjustable stop portion. As shown in FIG. 21, an annulus cutting tool handle 601 is provided with an elongate shaft assembly 602 with a proximal end 604 and a distal end 606 and a longitudinal axis 608. The elongate shaft assembly 602 includes an outer shaft 652 with a blade locator portion 610 and a bar-shaped blade holder portion 612 at the distal end 606. As shown in FIG. 22, the outer shaft 652 includes a longitudinal throughbore 654 with an interior threaded portion 656 in which an inner shaft 658 (shown in FIG. 23) is received.

As shown in FIG. 23, the inner shaft 658 has a proximal end 660 and a distal end 662 with an external threaded portion 664 and an elongate stop member 666 with a distal stop surface 638. The elongate stop member 666 extends into the outer shaft 652 from the distal end 606 thereof and is rotatably received thereby and is operably connected to the distal end of the inner shaft 658 for being driven for reciprocating linearly translationally thereby. The inner shaft 658 has an outer diameter sized and configured to permit the inner shaft 658 to move both rotationally and translationally through the throughbore 654 of the outer shaft 652. The inner shaft 658 is operably connected to an actuator portion in the form of an adjustment knob 650 at its proximal end 660. By rotating the adjustment knob 650, the inner shaft 658 is turned so that the threaded portion 664 is advanced or retracted along the threaded portion 656 of the throughbore 654 of the outer shaft 652. As this occurs, the inner shaft 658 and the stop portion 666 shift longitudinally relative to the outer shaft 652 and blade holder portion 612.

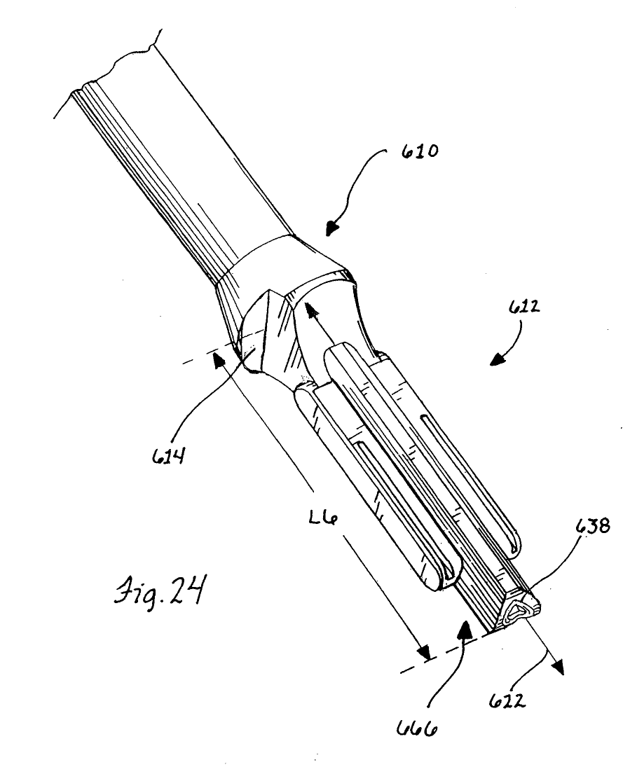

Thus, as shown in FIG. 24, rotation of the adjustment knob 650 allows adjustment of the distance L6 between the stop surface 638 and indentation 614, with distance L6 parallel to the longitudinal axis 622 of the blade holder portion 612. This will also change the distance between the distal tips 28 of the blades 10 and the stop surface 638 to change the size of the incision to be cut by the blades to a desired length. During surgery, the stop surface 638 contacts the annulus and is operable to limit at least a portion of the incision in the annulus to a predetermined size. For example, the stop surface 638 contacts the annulus to limit the length of the incision by limiting the portion of the length L1 of the cutting edge 18 of a surgical blade that pierces the annulus. Thus, the surgeon can easily adjust the cutting tool handle 601 so that the surgical blades cut the annulus at a desired predetermined length by rotating the adjustment knob 650 so that the stop surface 638 protrudes from the cutting tool 600 at a desired distance.

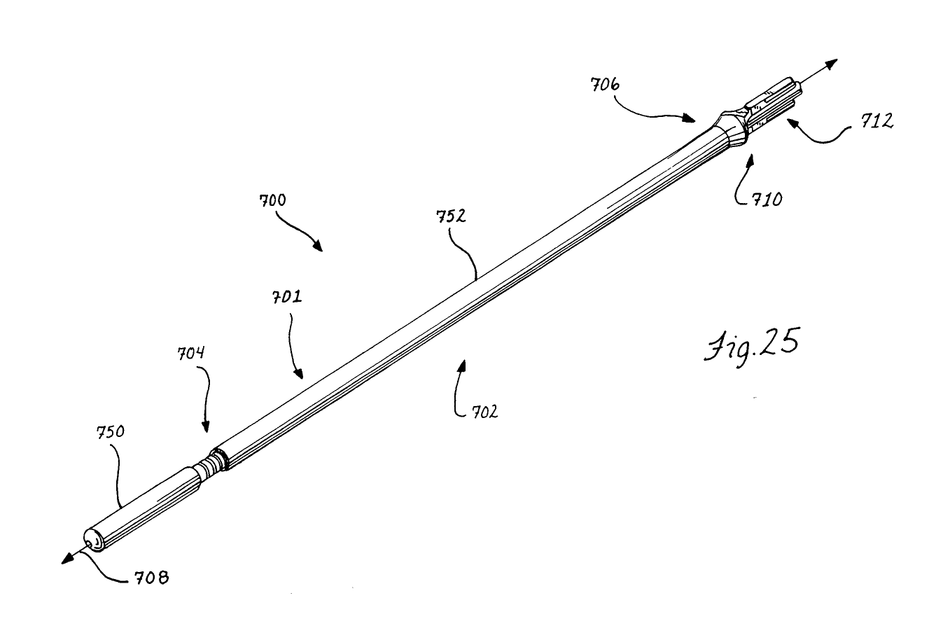

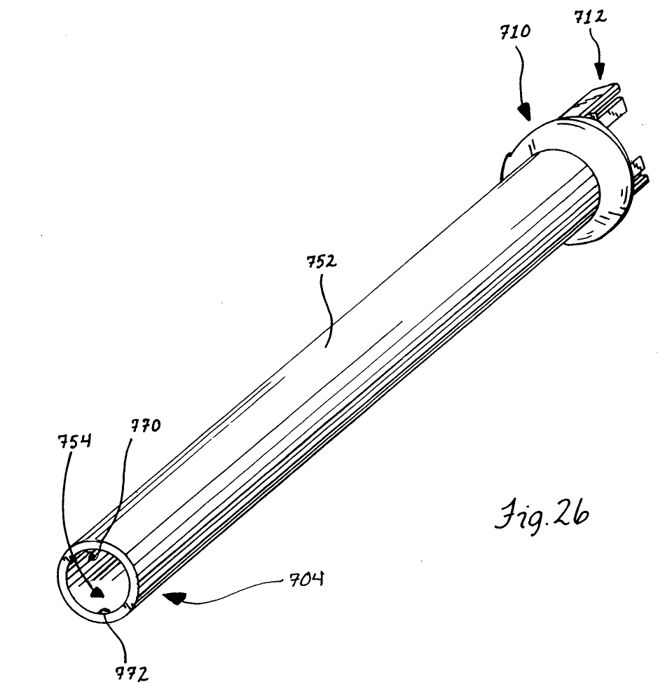

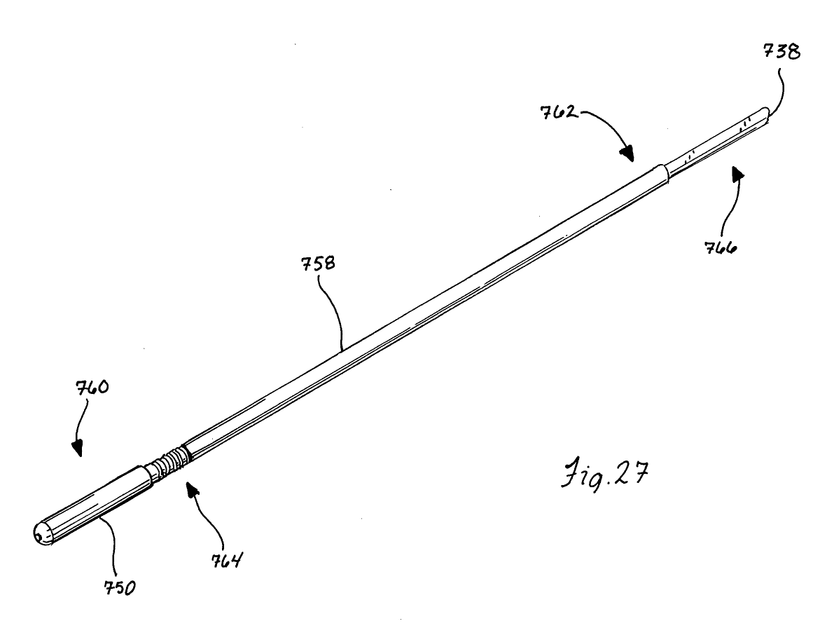

FIGS. 25-28 illustrate an alternative embodiment of a cutting tool with an adjustable stop portion using a detent mechanism to fix the stop surface at a selected position from a plurality of predetermined positions for the stop surface that may be selected. The cutting tool 700 includes cutting tool handle 701 having an elongate shaft assembly 702 with a proximal end 704 and a distal end 706 oriented about a longitudinal axis 708. The elongate shaft assembly 702 includes an outer shaft 752 with a blade locator portion 710 and a blade holder portion 712 at the distal end 706. As shown in FIG. 26, the outer shaft 752 includes a longitudinal throughbore 754 through which an inner stop shaft member 758 (shown in FIG. 27) is received. At least one, preferably at least two, detent members, such as resiliently mounted detent balls, are disposed on the inner surface of the proximal end 704 of the longitudinal throughbore 754. FIG. 26 shows two detent balls 770 and 772. While not illustrated, it should be noted that various other biasing mechanisms or detent devices such as spring-loaded or biased ball and detent catches or rings can be employed.

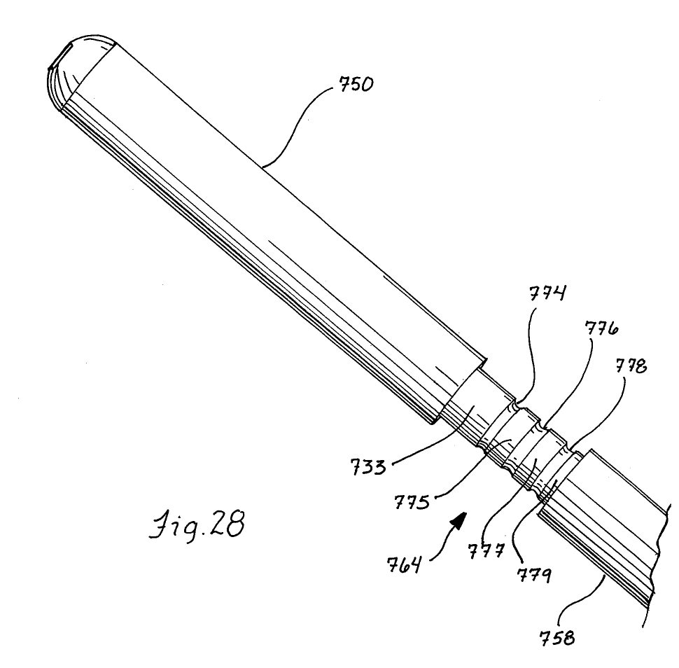

As shown in FIG. 27, the inner shaft 758 has a proximal end 760 with a grooved portion 764 comprising a plurality of recesses or grooves (774, 776, and 778 as shown in greater detail in FIG. 28) and a distal end 762 with an elongate stop portion 766 having a stop surface 738. The inner shaft 758 has an outer diameter sized to permit the inner shaft 758 to move translationally through the throughbore 754 of the outer shaft 752. The inner shaft 758 is operably or integrally connected to an actuator in the form of an enlarged knob 750 at its proximal end 760.

The application of force parallel to the longitudinal axis 708 of the elongate shaft portion 752 causes the inner shaft 758 to shift longitudinally in the outer shaft 752 with detent balls 770 and 772 being depressed by the raised collar portions 773, 775, 777, and 779 of the inner shaft 758 between which the annular grooves 774, 776, and 778 are formed. Once the desired position of the stop surface is obtained, the balls will seat in an aligned one of the grooves.

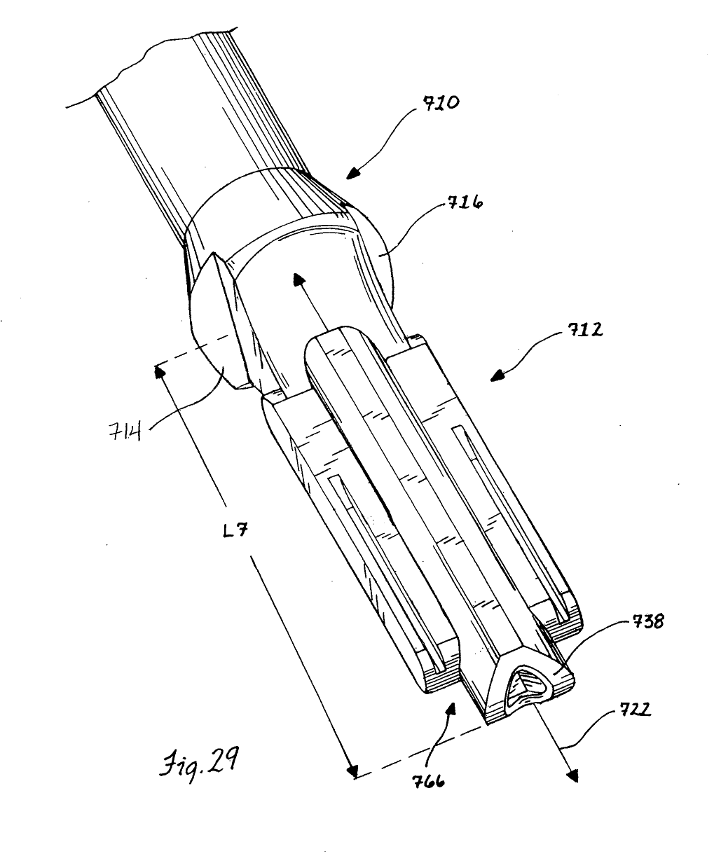

As shown in FIG. 29, longitudinal movement of the outer shaft 752 allows adjustment of the distance L7 between the stop surface 738 and indentation 714 (or indentation 716), with distance L7 being parallel to the longitudinal axis 722 of the blade holder portion 712. During surgery, the stop surface 738 contacts the annulus and is operable to limit at least a portion of the incision in the annulus wall to a predetermined size. For example, the stop surface 738 contacts the annulus to limit the length of the incision by limiting the portion of the length L1 of the cutting edge 18 of a surgical blade that pierces the annulus. Thus, the surgeon can easily adjust the cutting tool handle 701 so that the surgical blades cut the annulus at a desired length by moving the inner shaft 758 in the outer shaft 752 so that the stop surface 738 protrudes from the cutting device 700 at a desired distance.

A variety of surgical blades may be used with the cutting tools described herein. Generally, commercially available surgical blades may be easily mounted on the blade holders described herein. Custom surgical blades may also be used as long as the aperture and abutment surface of the custom surgical blades are compatible with the blade holder and blade locator portions of the cutting tools of the present invention.

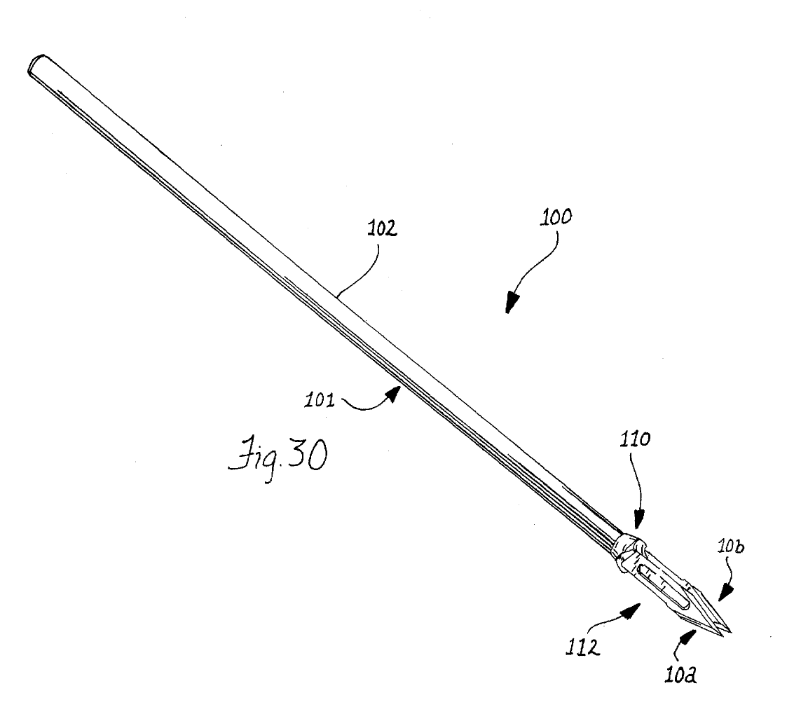

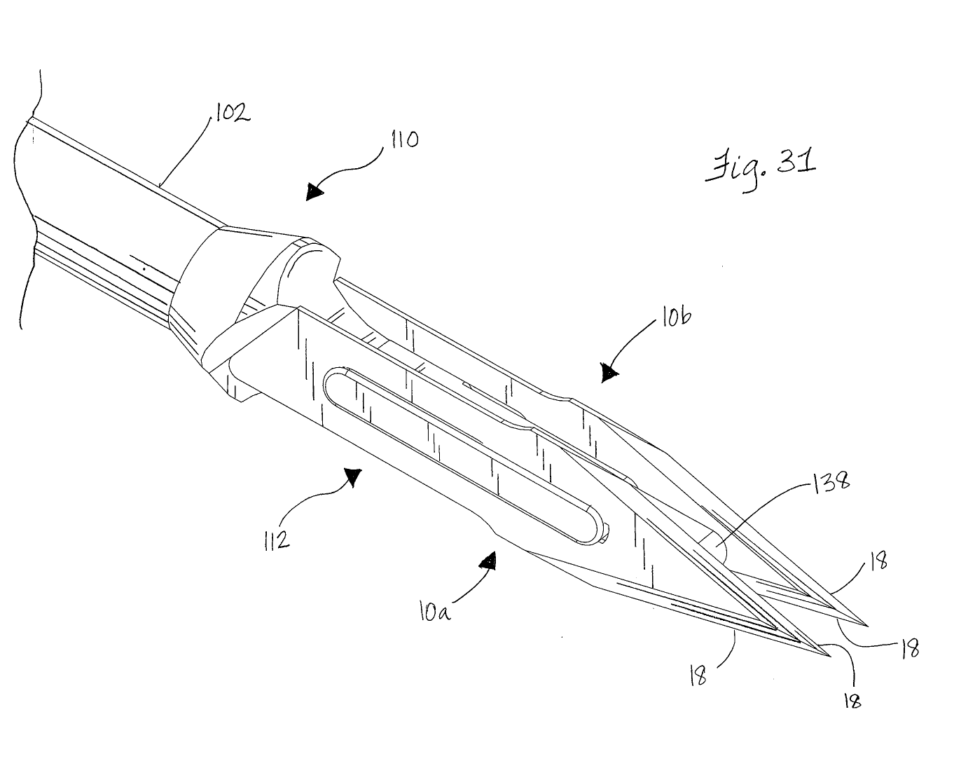

As shown in FIGS. 30 and 31, two custom surgical blades are mounted on the annulus cutting tool handle 101 of the cutting tool of FIG. 2. As best seen in FIG. 31, the custom surgical blades 10a and 10b each have two cutting edges 18 and are mounted on the cutting tool handle 101 as described above. The surgeon may create incisions in the annulus as previously described by inserting the custom surgical blades into the annulus to create two parallel incisions of identical length in a controlled manner. Once the surgeon has identified the appropriate location in the annulus for the incisions, the surgeon directs a desired amount of force to cutting tool handle 101 to guide the surgical blades into the annulus. The surgeon may continue to apply force until the stop surface 138 of the cutting tool handle 101 contacts the annulus. The stop surface 138 prevents further longitudinal movement of the surgical blades into the annulus, thus limiting the length of the incisions, as explained previously.

It should be noted that the cutting tools with an adjustable stop portion may be used in conjunction with any of the configurations of the blade locator portions and/or blade holder portions described herein.

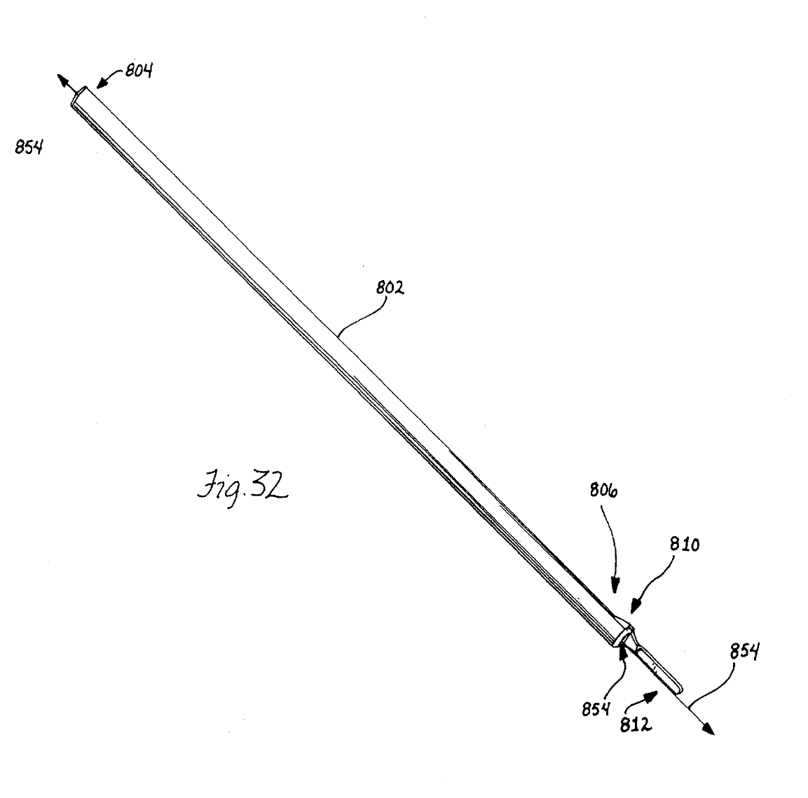

FIGS. 32-36 illustrate a cannulated cutting tool handle 801 of cutting tool 800 that is configured to hold a single surgical blade and may be used in conjunction with a guide wire. As shown in FIG. 32, the cutting tool handle 801 has an elongate handle portion 802 with a proximal end 804 and a distal end 806, the distal end 806 having a blade locator portion 810 for orientating a surgical blade and a blade holder portion 812 for attachment of the surgical blade to the cutting tool handle 801. The elongate handle portion 802 is cannulated so as to include a central longitudinal throughbore 854 extending from the elongate handle proximal end 804 to the elongate handle distal end 806.

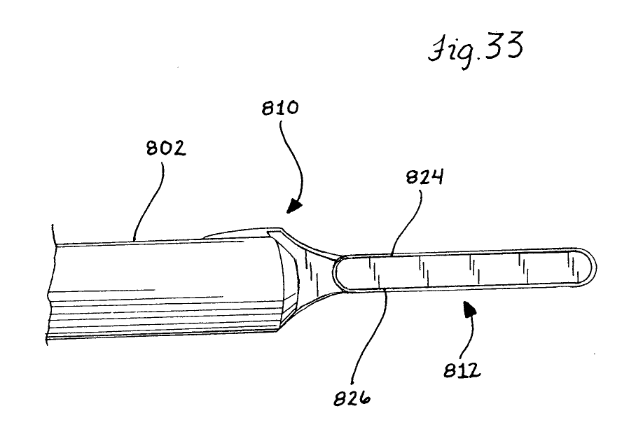

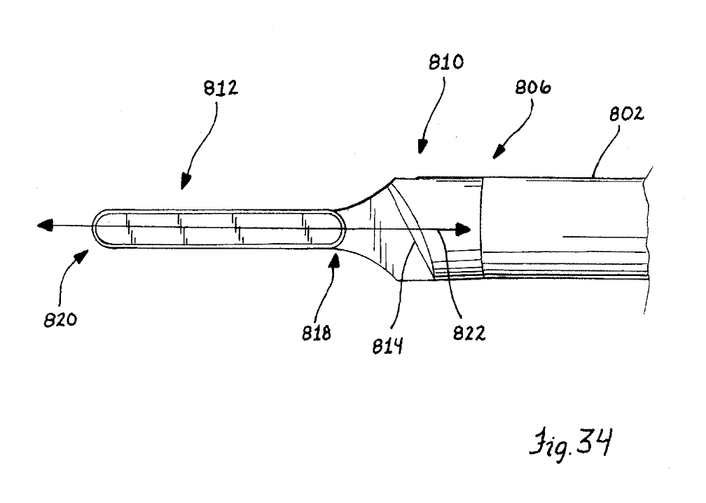

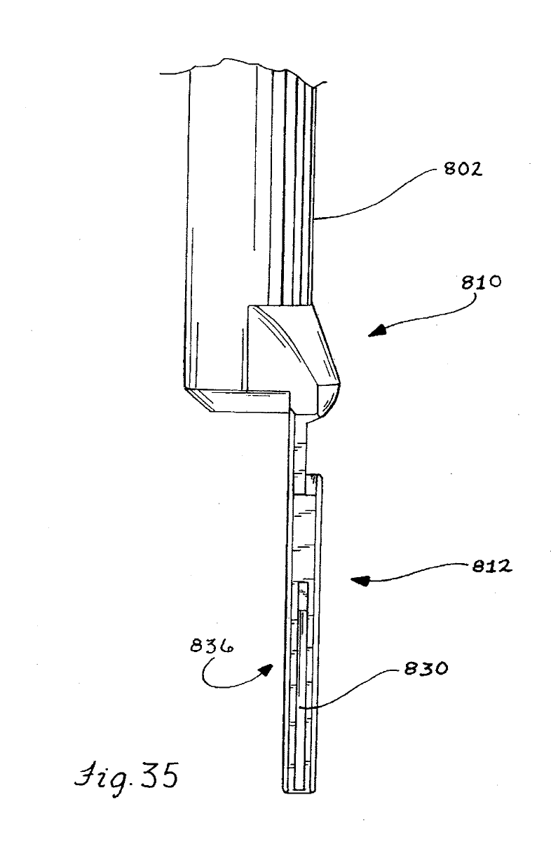

As best seen in FIGS. 33 and 34, the blade locator portion 810 is located adjacent the blade holder portion 812. As best shown in FIG. 34, the blade locator portion 810 includes a single indentation 814, the orientation of which determines the orientation of the surgical blade. The blade holder portion 812 has a proximal end 818, a distal end 820, two linear recesses or elongate grooves 830 and 836 (best shown in FIG. 35), and a longitudinal axis 822.

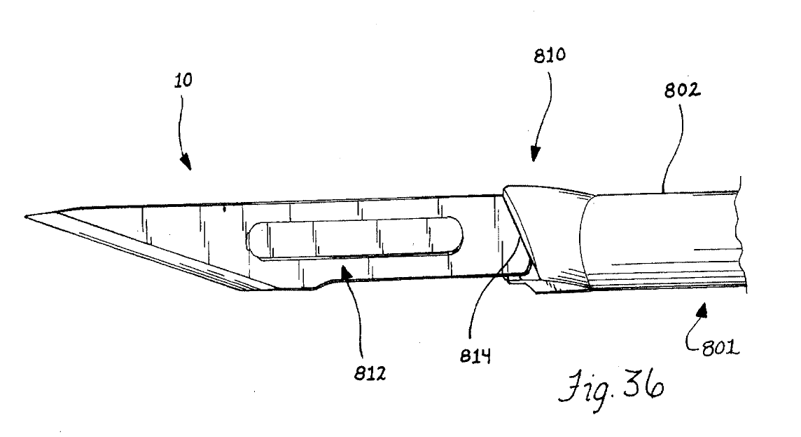

As shown in FIG. 36, a single surgical blade may be mounted on the cutting tool handle 801. A surgical blade with at least a partially curved cutting edge (such as a blade similar to that depicted in FIG. 1B) may be used with the cutting tool handle 801 to create an arcuate or substantially circular incision in the annulus.

The cutting tool handle 801 may also be used in conjunction with a guide wire. A surgeon may insert a guide wire into the annulus at the desired location for incision and drive the guide wire to a desired depth until secure in the annulus. As shown in FIG. 32, the distal end 806 of the handle 801 is positioned so that the opening at the distal end 806 of the handle 801 to the throughbore 854 is aligned with and can be guided onto the free end of the guide wire for advancing the handle 801 carrying the blade 10 along the guide wire towards the annulus. The surgeon may then make the predetermined incision in the annulus. In a preferable aspect, the incision is a circular incision in the annulus.

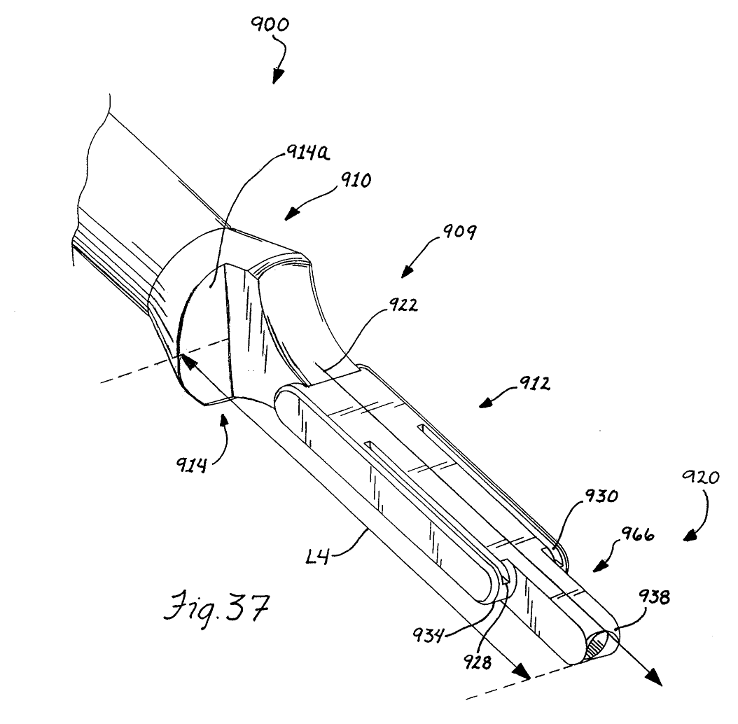

FIG. 37 illustrates an annulus cutting tool 900 having substantially the same structure as cutting tool 100 but having an alternative configuration of the blade mounting portion 909 over blade mounting portion 109. The blade mounting portion 909 includes a blade locator portion 910 and a bar-shaped blade holder portion 912. The blade locator portion 910 includes indentations 914 and 916 (not shown) including rear locating surfaces as previously described in regard to cutting tool 100, the orientation of which determines the orientation of the surgical blades. The distal end 920 of the blade holder portion 912 includes an elongate stop portion 966 including a stop surface 938 that extends for a sufficient distance transverse to the four elongate retaining grooves 928, 930, 934, and 936 (not shown) as illustrated so that the bar holder portion 912 cannot be pushed into the incisions, stopping continued cutting of the annulus wall with the blades.

As illustrated, the elongate stop portion 966 extends for a preset distance beyond the retaining grooves 928, 930, 934, and 936. The stop surface 938 is spaced from indentations 914 and 916 (not shown) by a fixed length L4 parallel to the longitudinal axis 922 of the blade holder portion 912. Since the stop surface 938 will be at a predetermined position recessed back from the blade distal tip of one or more cutting blades, this will allow the length of the incision made by the inclined cutting edges 18 thereof to be known depending on the inclination or declination of the cutting edges and their length relative to the location of the stop surface 938. During surgery, the stop surface 938 is the rounded, free end of the bar holder portion 912 and is sized so as to be operable to contact the annulus and limit the size of the incision made in the annulus made by the surgical blades to a predetermined length. For example, the stop surface 938 contacts the annulus to limit the length of the incision by limiting the portion of the length L1 of the cutting edge 18 that pierces and cuts the annulus.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. A cutting tool comprising:
a surgical blade having proximal and distal ends;
an elongate handle shaft having proximal and distal ends;
a blade holder portion at the handle distal end configured for mounting the surgical blade thereto;
a central throughbore extending through the handle shaft from the proximal end to the distal end thereof to permit a guide wire to be inserted therethrough.

2. The cutting tool of claim 1 wherein the surgical blade has an elongate slot and the blade holder portion is bar-shaped to fit in the blade slot for mounting the blade to the bar-shaped holder portion.

3. The cutting tool of claim 1 wherein the blade has opposing longitudinal edges on either side of the slot, and blade holder portion comprises at least two elongate grooves in which the blade edges fit for mounting the surgical blade to the blade holder portion.

4. The cutting tool of claim 1 wherein the handle includes a stop adjacent the blade holder portion that is operable to limit insertion of the at least one surgical blade into the annulus for defining a predetermined size for the incision in the annulus cut therewith.

5. The cutting tool of claim 1 wherein the shaft has a longitudinal axis, and the surgical blade has a cutting edge extending lengthwise obliquely to the shaft axis, the blade holder portion defines a predetermined fixed position of the surgical blade mounted thereto, and the stop is disposed at an intermediate position along the length of the cutting edge.

6. The cutting tool of claim 1 wherein the surgical blade comprises a pair of surgical blades, and the blade holder portion is configured to mount both of the surgical blades thereto.

7. The cutting tool of claim 6 wherein the blades of the blade holder portion are configured so that the blade holder portion mounts the blades to extend parallel to each other.

8. The cutting tool of claim 1 wherein the shaft includes a blade locator portion rearwardly of the blade holder portion configured for engaging the two surgical blades to provide each surgical blade with a predetermined orientation mounted to the blade holder portion at the handle distal end.

9. The cutting tool of claim 8 wherein the predetermined orientation of the blades is one of an identical orientation and a reversed orientation.

10. The cutting tool of claim 8 wherein the blade locator portion allows the blade to have either of two predetermined orientations when engaged therewith.

11. The cutting tool of claim 1 wherein the surgical blade comprises first and second blades, the elongate handle shaft comprises first and second handle shafts, and the blade holder portion comprising a first blade holder portion of the first shaft configured for mounting the first blade thereto and a second blade holder portion of the second shaft configured for mounting the second blade thereto with one of the shafts being hollow to allow the shaft to be slid therethrough to allow the blades to be advanced and retracted relative to each other for sequentially cutting incisions therewith.

12. The cutting tool of claim 11 wherein the first blade comprises a pair of parallel first blades mounted to the first blade holder portion and the second blade comprises a pair of parallel second blades mounted to the second blade holder portion orthogonal to the first blades for cutting a box-shaped opening in the annulus.

13. The cutting tool of claim 11 wherein the first blade comprises a pair of parallel first blades mounted to the first blade holder portion and the second blade comprises a single second blade mounted to the second blade holder portion for cutting a three-sided opening in the annulus so that there is a fourth uncut side that forms a flap portion of the annulus to cover the three-sided opening.

14. A cutting tool assembly for cutting an opening in an annulus of the spinal disc, the cutting tool assembly comprising:
a first cutting device having a first pair of surgical blades mounted thereto;
a second cutting device having a second pair of surgical blades mounted thereto; and
a sliding fit between the first and second cutting devices to allow the cutting devices to be advanced and retracted relative to each other for cutting incisions to form opposite sides of the annulus opening with one of the first and second pairs of surgical blades and the remaining opposite sides of the annulus opening with the other pair of surgical blades.

15. The cutting tool assembly of claim 14 wherein one of the cutting devices has a stop that is operable to limit insertion of the associated surgical blade into the annulus for defining a predetermined size for the incision in the annulus cut therewith.

16. The cutting tool of claim 14 wherein the first and second cutting devices each include an elongate shaft with one of the shafts being hollow to slidably receive the other therein for advancing and retracting the cutting blades relative to each other.

17. The cutting tool of claim 16 wherein each shaft includes a distal blade mounting portion configured for mounting two surgical blades thereto with the blade mounting portion of the hollow shaft having a split configuration to fit about the other blade mounting portion.

18. A method of cutting an opening in an annulus of a spinal disc, the method comprising:
inserting a pair of surgical blades mounted to a cutting tool into the annulus;
cutting the annulus with the pair of surgical blades to form two sides of the annulus opening;
shifting the cutting tool so that the pair of surgical blades cut the remaining two sides about the opening to complete the cutting of the opening in the annulus.

19. The method of claim 18 further including mounting the pair of surgical blades to the cutting tool to extend parallel to each other so that the annulus is cut with the blades to form two parallel sides of the annulus opening.

20. The method of claim 18 wherein shifting the cutting tool comprises retracting the pair of surgical blades from the annulus, orienting the pair of surgical blades so that the surgical blades are perpendicular to the cut sides of the annular opening, advancing the pair of surgical blades into the annulus, and cutting the annulus with the pair of surgical blades to form parallel incisions that interconnect the previously cut parallel sides of the annular opening.

21. The method of claim 18 wherein shifting the cutting tool comprises turning the pair of surgical blades in the annulus to form an opening having a substantially curved perimeter.

22. The method of claim 18 wherein inserting a pair of surgical blades into the annulus comprises advancing the pair of surgical blades into the annulus until a stop of the cutting tool contacts the annulus to define the depth of insertion of cutting edges of the blades into the annulus.

23. The method of claim 22 further including adjusting the position of the stop of the cutting tool.

24. The method of claim 18 further comprising securing one end of a guide wire at a desired position in the annulus, guiding the cutting tool onto the free end of the guide wire and advancing the cutting tool along the guide wire towards the annulus.
